(19) United States
(12) Patent Application Publication
(10) Pub. No.: US 2021/0268023 A1
(43) Pub. Date: Sep. 2, 2021

(54) Enhanced CAR Tregs and Bi-Specific Antibodies for Induction of Immune Tolerance, Treating Autoimmune Diseases and Preventing Transplantation Rejection

(71) Applicant: THE TRUSTEES OF COLUMBIA UNIVERSITY IN THE CITY OF NEW YORK, New York, NY (US)

(72) Inventors: Megan Sykes, New York, NY (US); Mohsen Khosravi Maharlooei, Fort Lee, NJ (US); Remi Creusot, New York, NY (US)

(21) Appl. No.: 17/188,640

(22) Filed: Mar. 1, 2021

Related U.S. Application Data
(60) Provisional application No. 62/983,715, filed on Mar. 1, 2020.

(51) Int. Cl. A61K 35/17; A61P 37/02; C07K 14/74; C07K 14/725; A61K 45/06

(52) U.S. Cl. CPC A61K 35/17; A61P 37/02; C12Y 304/22; C07K 14/7051; A61K 45/06; C07K 14/70539

(57) ABSTRACT

The present disclosure provides for conversion-resistant CAR regulatory T cells (Tregs) and bi-specific antibodies, and methods to use these Tregs and antibodies for the treatment of autoimmune diseases and for prevention of organ transplant rejection.

### CROSS-REFERENCE TO RELATED APPLICATION

The present application claims priority to U.S. Provisional Patent Application No. 62/983,715 filed on Mar. 1, 2020, which is incorporated herein by reference in its entirety.

### FIELD OF THE INVENTION

The present disclosure provides for conversion/conditioning-resistant CAR regulatory T cells (Tregs), in addition to bi-specific antibodies, and methods to use these Tregs and antibodies for the treatment of autoimmune diseases and for prevention of organ transplant rejection.

### BACKGROUND

Suppression of the immune system is beneficial in organ transplantation and treatment of autoimmune disorders. Organ transplantation has emerged as a preferred method of treatment for many forms of life-threatening diseases that involve organ damage. However, transplantation rejection may occur when an organism receiving transplanted cells or tissue mounts an undesired immune response to that tissue.

Regulatory T (Treg) cells are immunosuppressive and generally suppress or downregulate induction and proliferation of effector T (Teff) cells. Tregs prevent immune responses to non-pathogenic antigens, and are the primary modulators of peripheral tolerance. Treg cells can be tuned to tolerate select antigens through exposure to these stimuli in vivo or ex vivo. Treg therapy is also a promising approach to restore immune tolerance in patients with autoimmune diseases.

Despite all advances in developing immunosuppressive medications, immune responses in autoimmunity and transplantation are difficult to control without excessive toxicity (Sachs 2018; Kawai et al. 2008; Yamada et al. 2017; Bandgar et al. 2013). Treg therapy has been widely used for control of immune responses in different models of autoimmunity and transplantation (Bluestone et al. 2015; Romano et al. 2019; Tang and Bluestone 2013; Sharabi et al. 2018). It is well known that compared to non-specific Tregs, antigen-specific Tregs are more potent in suppressing immune responses and they harbor a lower risk of general immunosuppression (Haddadi et al. 2020; Adair et al. 2017). In order to generate antigen-specific Tregs, patient's Tregs can be virally transduced with specific T cell receptors (TCRs) or chimeric antigen receptors (CARs). As TCRs recognize antigens in the context of MHCs, patient-specific TCRs need to be generated according to patient's MHCs, while CARs only recognize the antigen and can be used universally. Chimeric antigen receptor (CAR) Tregs against HLA-A2 have been shown to be effective in preventing xeno-graft-versus-host disease caused by HLA-A2-restricted T cells in a human PBMC-transferred NSG mouse model (MacDonald et al. 2016). HLA-A2-CAR Tregs were also effective in controlling allogeneic responses in a skin transplantation model in humanized (HU) mice (Dawson et al. 2019). CAR Tregs were shown to be effective in controlling immune reactions in some autoimmune disease models (Elinav et al. 2008; Skuljec et al. 2017; Fransson et al. 2012).

Two recent major advancements in designing CAR constructs have opened new doors in developing strategies to improve the functionality and safety of CAR therapies. The first advancement introduced an on and off switch for temporal control over CAR T cell action (Chia-Yun et al. 2015). Development of split, universal and programmable (SUPRA) CARs, in which the target modules and the CARs are separated and bind to each other through a zipper molecule (such as leucine zipper) makes it possible to switch targets without re-engineering the Tregs (Cho et al. 2018). Different zipper molecules with a range of variable affinities provide the opportunity to evaluate the effect of Treg activation strength on functionality of CAR Tregs.

CAR Treg cell therapy is promising for preventing and treating autoimmune diseases and promoting immunologic tolerance in transplantation by recognizing non-threatening antigens. However, clinical implementation of Treg cell therapy is hindered by multiple factors, including CAR-Treg plasticity and inefficient trafficking to target organs and their draining lymph nodes. As CAR Tregs are specific for self-antigens, they could have detrimental effects if they lose their regulatory phenotype and convert to effector CAR T cells. Despite promising results in using CAR Tregs for tolerance induction, there are still major considerations regarding the plasticity of CAR Tregs (Koenen et al. 2008) and the effect of T cell-depleting conditioning regimens (Neelapu et al. 2019; Mancusi et al. 2019) on these cells that need to be addressed before their clinical application. Inflammation is shown to drive Treg conversion (Hua et al. 2018). CAR Tregs can quickly reject the target organs if they lose their regulatory phenotype and convert to Teff.

### SUMMARY

The present disclosure provides approaches to prevent conversion of CAR Tregs and to induce apoptotic cell death or restrain the antigen specificity in CAR Tregs that have converted to effector T (Teff) cells.

In order to make CAR Tregs resistant to a T cell-depleting conditioning regimen involving an anti-CD2 antibody, a method is introduced which utilizes CRISPR technology to remove the CD2 molecule from CAR Tregs genome. This will ensure that these Tregs do not get affected by this antibody which will be used to deplete recipient T cells in order to open space for the adoptively-transferred CAR Tregs. Additionally, an approach is introduced to boost the immunosuppressive effect of CAR Tregs through targeted secretion of specific immunomodulatory molecules upon interaction of CAR Tregs with their target antigens in the sites of transplantation and autoimmunity. These immunomodulatory molecules will suppress local innate and adaptive immune cells, including T cells, B cells, monocytes and macrophages. This will help to improve the efficiency of CAR Tregs in suppressing unwanted immune responses. In order to re-direct CAR Tregs and other non-specific Tregs to the sites of transplantation and autoimmunity, two approaches are presented, which include an engineering approach to introduce chemokine receptors to CAR Tregs that re-direct them to the tissues of interest and also the use of a bi-specific antibody with specificities toward the target tissue and a Treg marker such as CTLA-4 that re-directs both CAR Tregs and non-specific Tregs to the sites of transplantation and autoimmunity.

The present disclosure provides the basis for conversion/conditioning-resistant CAR Tregs for induction of immune tolerance in transplantation and for treating/preventing autoimmune diseases. The present compositions and methods prevent conversion of CAR Tregs to Teff cells, by, e.g., inducing cell death or restraining the antigen specificity of the CAR Tregs that have converted to Teff cells. The conversion/conditioning-resistant CAR Tregs cells may be used for local delivery of immunomodulatory molecules in transplantation and autoimmunity. The long-term maintenance of Treg activity may be ensured by overexpressing regulatory factors that prevent the unwanted and potentially dangerous conversion of Treg cells.

The present cell/composition may be used in various therapeutic, prophylactic, diagnostic and other methods. The present cells/compositions and methods may be used to reduce complications associated with organ or tissue transplantation. The present disclosure also provides for compositions and methods for reducing the likelihood of transplant rejection, treat transplant rejection, inducing immunosuppression, and/or treating an autoimmune disorder. The compositions contain the present cells.

Trafficking CAR Tregs into the target tissues and their draining lymph nodes is of significant importance for efficient function of CAR Tregs. The present disclosure also provides methods of combining CAR Treg therapy with administration of bispecific antibodies that recognize Tregs and target tissues to improve trafficking of CAR Tregs to the target sites and their draining lymph nodes. Additionally, it provides methods for engineering of CAR Tregs with chemokine receptor genes that re-direct the CAR Tregs to the sites of transplantation and autoimmunity.

The present Treg cells may be used to suppress rejection responses to donor organs where the specific antigens are unknown.

The present compositions and methods may be used for treating or preventing organ transplant rejection, and/or graft-versus-host diseases.

In some embodiments, the present disclosure provides for a Treg cell comprising a first nucleic acid construct encoding a CAR. The CAR comprises an antigen-binding region.

In some embodiments, the CAR binds to human leukocyte antigen A2 (HLA-A2).

In some embodiments, the CAR may be operably linked to a Treg-specific promoter including but not limited to the forkhead box P3 (Foxp3) promoter. Other Treg-specific genes include but are not limited to peptidase inhibitor 16 (Sadlon et al. 2010).

In some embodiments, the Treg cell may further comprise a suicide gene, such as an inducible suicide gene including but not limited to a suicide gene that encodes Caspase 3, Caspase 7, Caspase 8 or Caspase 9, or encodes a chimeric protein comprising CD8 and Caspase 8.

In some embodiments, the suicide gene is operably linked to a Teff-specific promoter, including but not limited to a promoter of granzyme B, perforin, IL-17 or IL-7 receptor α (CD127) genes.

The antigen-binding region of the CAR may be a single-chain variable fragment (scFv) comprising a light chain variable region (V_{L}) and a heavy chain variable region (V_{H}).

In some embodiments, the CAR comprises a cytoplasmic signaling domain of CD3ζ.

In certain embodiments, the Treg cell is substantially devoid of CD2.

The Treg cell may further comprise a second nucleic acid construct encoding one or more immunomodulatory molecules. In some embodiments, the second nucleic acid construct is operably linked to a nuclear factor of activated T cells (NFAT)-responsive promoter. Immunomodulatory molecules include but are not limited to PD-L1, TGF-β, CTLA-4Ig, IL-10, IDO1, an anti-CD40 antibody, an anti-IFNγ antibody and combinations thereof.

In some embodiments, the second nucleic acid construct will contain genes that enhance the engraftment of hematopoietic stem cells (HSCs). HSC engraftment-enhancing molecules include but are not limited to, SCF, FLT3L, thrombopoietin, CXCL12, and combinations thereof. This strategy will be used for improving mixed hematopoietic chimerism as a method for induction of immune tolerance in transplantation. In some embodiments, it can be used in autologous HSC transplantation as a method for treatment of autoimmune diseases.

The present disclosure also provides for methods of using the disclosed cells and compositions.

The present disclosure also provides for compositions, including pharmaceutical composition, comprising the cells disclosed herein as well as kits comprising cells, compositions and pharmaceutical compositions disclosed herein.

### BRIEF DESCRIPTION OF THE FIGURES

FIG. 1 shows the CAR constructs designed to study CAR Treg conversion.

FIG. 2 shows the expansion of polyclonal and allospecific Tregs from humanized mice. At week 15 after generation of humanized mice, CD4⁺CD25^{high}CD127^{low} Tregs were sorted from splenocytes and were polyclonally expanded by anti-CD3/CD28 beads or by allogeneic activated B cells. Both types of Tregs could be expanded ex vivo after three days (left). Suppressive function of these types of Tregs was determined in an assay using B cells that were used to expand the Tregs as stimulator and splenic T cells from other humanized mice of the same cohort (right).

FIGS. 3A-3E show historical humanized mouse models of Type 1 diabetes (T1D). FIG. 3A) Representative luciferase imaging of a mouse transplanted with 3000 embryonic stem cells (ES)-derived beta cells. FIG. 3B) Human c-peptide levels in the serum of humanized mice with HLA-A2+ immune system transplanted with HLA-A2+ ES-beta cells and injected with TCR-Tg T cells. FIG. 3C) The proportion of human T cells with the 4 indicated transgenic TCRs among total T cells in spleen, blood and the muscle tissue, where the ES-beta cells were grafted. FIG. 3D) NSG RIP-DTR mice were either unreconstituted (naive) or received human fetal thymus grafts and HSCs (HU/HU). Six weeks later, they received 2000 islet equivalent (IEQ) allogeneic human islets under the kidney capsule followed 13 weeks later by treatment with 50 ng diphtheria toxin to kill the endogenous pancreatic beta cells. FIG. 3E) Islet grafts in HU/HU mice were heavily infiltrated with human leukocytes that included CD4 and CD8 T cells and human macrophages.

FIG. 4 shows schematic presentation of events in generation of humanized mouse models of T1D.

FIG. 5 shows schematics summarizing the present approach. FIG. 5A shows schematic presentation of events after CAR Treg encounters its target antigen. FIG. 5B shows schematic presentation of events when CAR Treg converts to effector CAR T cells. FIG. 5C shows schematic presentation of engineering approaches needed to generate enhanced conversion/conditioning-resistant CAR Treg.

### DETAILED DESCRIPTION

The present disclosure provides for CAR Tregs.

Genetic engineering approaches are used to induce apoptotic cell death and/or block the CAR expression in CAR Tregs that convert to Teff. HLA-A2 CAR Tregs are used to control autoimmune responses against HLA-A2+ human islets in a humanized mouse model of Type 1 diabetes (T1D) (both transplantation and autoimmunity models).

CRISPR screening studies could potentially uncover unknown biological aspects of Treg functionality and plasticity and reveal genes/pathways that could be clinically targeted to improve Treg function. Using CAR Tregs to deliver immunomodulatory molecules will enhance their immunosuppressive capacity. Local delivery of other factors to control other biological functions with this system may also be achieved. Local of delivery of factors supporting HSC engraftment is used to improve mixed chimerism for induction of immune tolerance to donor antigens in transplantation. This will improve the results of transplantation through induction of tolerance to donor antigens.

Additionally, while lymphodepleting conditioning regimens are needed to open some space for adoptively-transferred CAR Tregs, they could potentially be toxic to these cells (Neepalu et al. 2019; Mancusi et al. 2019). In order to generate conditioning-resistant CAR Tregs, CRISPR is used to remove the CD2 molecule in these cells in order to make them resistant to a conditioning regimen consisting of anti-CD2 antibody, which has been successfully used in induction of mixed chimerism in patients receiving combined kidney and bone marrow transplantation (Kawai et al. 2008). This would allow CAR Tregs to persist and expand in a lymphopenic environment after anti-CD2 injection.

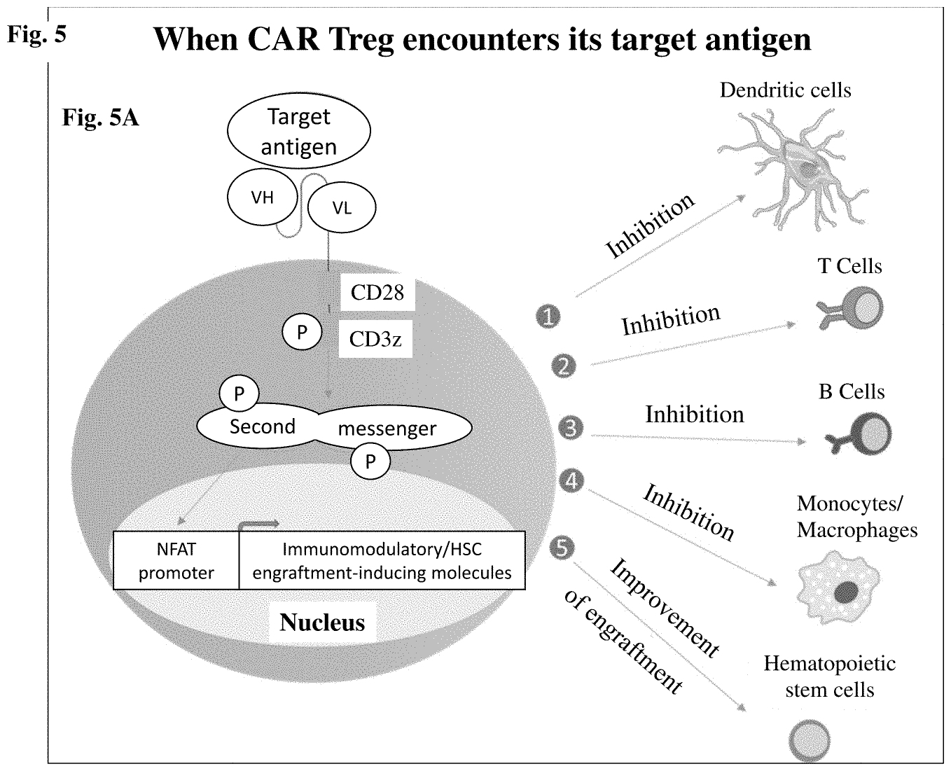

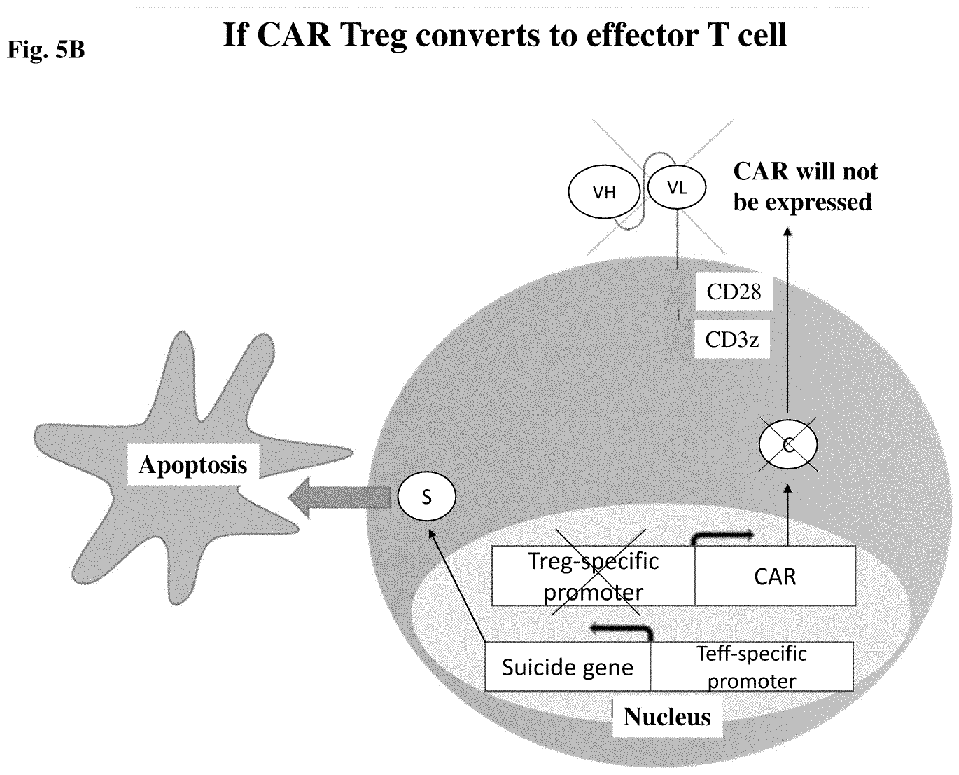

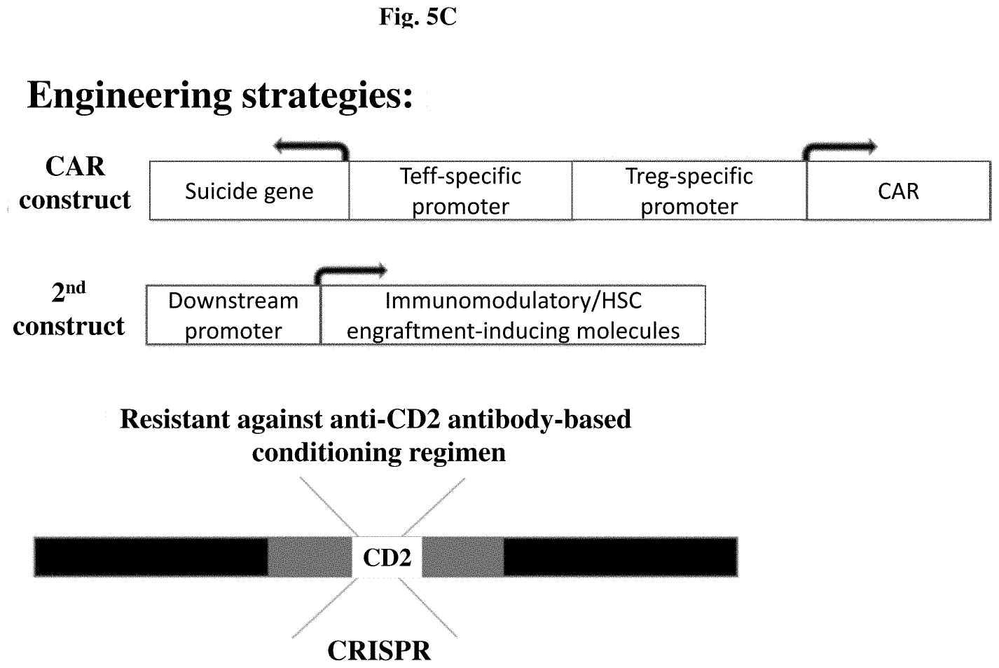

The present disclosure provides for conversion-resistant CAR Tregs to induce immune tolerance in transplantation and for treating/preventing autoimmune diseases. The present compositions and methods prevent transformation of CAR Tregs to effector T (Teff) cells, by, e.g., inducing cell death or restraining the antigen specificity of the CAR Tregs that have converted to Teff cells. The conversion-resistant CAR Treg cells may be used for local delivery of immunomodulatory molecules in xenotransplantation and autoimmunity. The long-term maintenance of Treg activity may be ensured by overexpres sing regulatory factors that prevent the unwanted and potentially dangerous conversion of Treg cells. See FIG. 5.

The present cell/composition may be used in various therapeutic, prophylactic, diagnostic and other methods. The present cells/compositions and methods may be used to reduce complications associated with organ or tissue transplantation. The present disclosure also provides for compositions and methods for reducing the likelihood of transplant rejection, treat transplant rejection, inducing immunosuppression, and/or treating an autoimmune disorder. The compositions contain the present cells.

Trafficking CAR Tregs into the target tissues and their draining lymph nodes are of significant importance for efficient function of CAR Tregs. The present disclosure also provides methods of combining CAR Treg therapy with administration of bispecific antibodies that recognize Tregs and target tissues to improve trafficking of CAR Tregs to the target sites and their draining lymph nodes.

The present Treg cells may be used to suppress rejection responses to donor organs where the specific antigens are unknown.

The present compositions and methods may be used for treating or preventing organ transplant rejection, and/or graft-versus-host diseases.

The present disclosure provides for a Treg cell comprising a first nucleic acid construct encoding a CAR. The CAR comprises an antigen-binding region.

The first nucleic acid construct encoding the CAR may be operably linked to a Treg-specific promoter, such as the forkhead box P3 (Foxp3) promoter. Other Treg-specific genes include but are not limited to peptidase inhibitor 16 (Sadlon et al. 2010).

The CAR may bind to human leukocyte antigen A2 (HLA-A2). In some embodiments, the CAR binds to tissue specific antigens involved in autoimmune diseases.

The Treg cell may further comprise a suicide gene, such as an inducible suicide gene. The suicide gene may encode Caspase 3, Caspase 8 or Caspase 9. The suicide gene may encode a chimeric protein comprising CD8 and Caspase 8.

The suicide gene may be operably linked to a promoter of IL-7 receptor a (CD127) or other Teff-specific promoters, such as a promoter of granzyme B, perforin or IL-17 genes.

The antigen-binding region of the CAR may be a single-chain variable fragment (scFv) comprising a light chain variable region (V_{L}) and a heavy chain variable region (V_{H}).

In one embodiment, the CAR comprises a cytoplasmic signaling domain of CD3ζ.

In certain embodiments, the Treg cell is substantially devoid of a cell surface marker, including but not limited to CD2. Treg cell substantially devoid of CD2 can be conditioning-resistant where they are resistant to a conditioning regimen using an anti-CD2 antibody to, e.g., induce mixed chimerism in patients receiving combined kidney and bone marrow transplantation.

The Treg cell may further comprise a second nucleic acid construct encoding an immunomodulatory molecule.

The second nucleic acid may be operably linked to a nuclear factor of activated T cells (NFAT)-responsive promoter.

Non-limiting examples of the immunomodulatory molecules include PD-L1, TGF-β, CTLA-4Ig, IL-10, IDO1, an anti-CD40 antibody, and an anti-IFNγ antibody. Non-limiting examples of the HSC engraftment-enhancing molecules include SCF, FLT3L, thrombopoietin, CXCL12, and combinations thereof.

The present disclosure provides for a regulatory (Treg) cell comprising a first nucleic acid construct encoding a chimeric antigen receptor (CAR) comprising an antigen-binding region. The cell may be substantially devoid of endogenous T-cell receptors (TCRs).

The antigen-binding region may comprise a light chain variable region (V_{L}) and a heavy chain variable region (V_{H}). The antigen-binding region may comprise a single-chain variable fragment (scFv).

The method may further comprise administering a bispecific antibody, such as a bispecific antibody specific to the cell (e.g., the Treg cell) and a target site. In one embodiment, the bispecific antibody is specific to CTLA-4 and an islet surface antigen.

The present disclosure provides for a method of treating or preventing an autoimmune disease in a subject. The method may comprise administering the cell/composition to the subject. The autoimmune disease may be Type 1 diabetes (T1D) or other autoimmune diseases.

Also encompassed by the present disclosure is a method of inducing immune tolerance, or treating or preventing rejection, in a subject to a graft obtained from a donor mammal. The method may comprise administering the present cell/composition to the subject before, during and/or after transplantation.

The graft may comprise cells, a tissue or an organ. For example, the graft may comprise a heart, a kidney, a liver, a pancreas, a lung, an intestine, skin, a small bowel, a trachea, a cornea, or combinations thereof.

The method may further comprise administering an immunosuppressant to the subject.

The present disclosure also provides for a kit comprising the present cell/composition.

In certain embodiments, to enable temporal control over CAR Treg cells, the CAR contains zipper molecules.

In one embodiment, the present disclosure provides for a method of treating or ameliorating graft-versus-host disease and/or transplant rejection in a subject comprising administering to the treated subject a composition comprising the present cells in an amount sufficient to decrease one or more of the symptoms of graft-versus-host disease and/or transplant rejection in the subject.

In another embodiment, the present cells are administered to a subject having an inflammatory disease or an immune disorder such as an autoimmune disease.

The disclosure features methods of reducing the likelihood of transplant rejection, treating transplant rejection, inducing immunosuppression, and/or treating an autoimmune disorder in a subject by administering to the subject the present cells in an effective amount.

In addition to the present cells, the present methods may further comprise administering a second therapeutic agent such as an immunosuppressant, a tumor necrosis factor antagonist (a TNF-antagonist), a CTLA4-antagonist, an anti-IL-6 receptor antibody, an anti-CD20 antibody, or a combination thereof.

### Regulatory T-Cells (Tregs)

Natural regulatory T-cells (Tregs) are CD4⁺CD25⁺FOXP3⁺ T lymphocytes that control innate and adaptive immune responses. Natural Tregs also express low amounts of CD127, develop in the thymus, express GITR and CTLA-4. Tregs suppress effector T (Teff) cells from destroying their (self-) target, either through cell-cell contact by inhibiting T cell help and activation, through release of immunosuppressive cytokines such as IL-10 or TGF-β, through production of cytotoxic molecules such as granzyme B, through depleting IL-2 levels, or by changing the availability of specific nutrients in tissues.

Tregs can be genetically modified using recombinant techniques. Targeted or untargeted gene knockout methods can be used to engineer subject Tregs ex vivo prior to infusion into the subject. For example, the target DNA in the genome can be manipulated by deletion, insertion, and/or mutation using retroviral insertion, artificial chromosome techniques, gene insertion, random insertion with tissue specific promoters, gene targeting, transposable elements and/or any other method for introducing foreign DNA or producing modified DNA/modified nuclear DNA. Other modification techniques include deleting DNA sequences from a genome and/or altering nuclear DNA sequences. Nuclear DNA sequences, for example, may be altered by site-directed mutagenesis. Such methods generally use host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals. For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

Similarly, the CRISPR-Cas system can be used for precise editing of genomic nucleic acids (e.g., for creating null mutations). In such embodiments, the CRISPR guide RNA and/or the Cas enzyme (e.g., Cas9) may be expressed. Similar strategies may be used (e.g., designer zinc finger, transcription activator-like effectors (TALEs) or homing meganucleases). Such systems are well-known in the art (see, for example, U.S. Pat. No. 8,697,359; Sander and Joung (2014) Nat. Biotech. 32:347-355; Hale et al. (2009) Cell 139:945-956; Karginov and Hannon (2010) Mol. Cell 37:7; U.S. Pat. Publ. 2014/0087426 and 2012/0178169; Boch et al. (2011) Nat. Biotech. 29:135-136; Boch et al. (2009) Science 326:1509-1512; Moscou and Bogdanove (2009) Science 326:1501; Weber et al. (2011) PLoS One 6:e19722; Li et al. (2011) Nucl. Acids Res. 39:6315-6325; Zhang et al. (2011) Nat. Biotech. 29:149-153; Miller et al. (2011) Nat. Biotech. 29:143-148; Lin et al. (2014) Nucl. Acids Res. 42:e47). Such genetic strategies can use constitutive expression systems or inducible expression systems according to well-known methods in the art.

As described herein and in some embodiments, Tregs are administered to a subject. Thus, the Tregs will have an immunocompatibility relationship to the subject and any such relationship is contemplated for use according to the present methods. For example, the Tregs can be syngeneic. The term “syngeneic” can refer to the state of deriving from, originating in, or being members of the same species that are genetically identical, particularly with respect to antigens or immunological reactions. Thus, the Tregs may be from a donor to a recipient who is genetically identical to the donor or is sufficiently immunologically compatible as to allow for transplantation without an undesired adverse immunogenic response. The Tregs may be autologous if the transferred cells are obtained from and administered to the same subject. The Tregs may be the subject's own cells which are harvested from, modified, and reinfused to the subject. The Tregs may be allogeneic where the cells are from a different animal/individual of the same species as the individual to whom the cells are introduced. Two or more individuals are said to be allogeneic to one another when the genes at one or more loci are not identical.

In addition, Tregs can be obtained from a single source or a plurality of sources (e.g., a single subject or a plurality of subjects). A plurality refers to at least two (e.g., more than one).

### Immune Cells Comprising CAR(s)

Chimeric antigen receptor (CAR) T cells are widely used to recognize antigens on cells with both high affinity and specificity and without the requirement for accessory recognition molecules, such as HLA antigens to “present” peptides. The T cell receptor of a CAR T cells is “swapped” with an antigen-binding heavy and light chains, thereby obviating the need for HLA accessory molecules.

In particular aspects, the immune cells are T cells or Treg cells that express a CAR.

A CAR is an artificially constructed hybrid protein or polypeptide typically containing an extracellular antigen binding domain and a transmembrane domain. The recombinant CAR may or may not be fused to signaling domains leading to activation of the T cell upon binding of the CAR to its target antigen. Characteristics of CARs include their ability to redirect T-cell specificity and reactivity toward a selected target in a non-MHC-restricted manner, exploiting the antigen-binding properties of monoclonal antibodies. The non-MHC-restricted antigen recognition gives T cells expressing CARs the ability to recognize antigen independent of antigen processing. Moreover, when expressed in T-cells, CARs advantageously do not dimerize with endogenous T cell receptor (TCR) alpha and beta chains.

In one embodiment, the stimulatory molecule is the zeta chain associated with the T cell receptor complex. In one aspect, the cytoplasmic signaling domain further comprises one or more functional signaling domains derived from at least one costimulatory molecule as defined below. The costimulatory molecule may also be 4-1BB (i.e., CD137), CD27 and/or CD28 or fragments of those molecules. In another aspect, the CAR comprises a chimeric fusion protein comprising an extracellular antigen recognition domain, a transmembrane domain and an intracellular signaling domain comprising a functional signaling domain derived from a stimulatory molecule. In one embodiment, the CAR comprises a chimeric fusion protein comprising an extracellular antigen recognition domain, a transmembrane domain and an intracellular signaling domain comprising a functional signaling domain derived from a co-stimulatory molecule and a functional signaling domain derived from a stimulatory molecule. Alternatively, the CAR may comprise a chimeric fusion protein comprising an extracellular antigen recognition domain, a transmembrane domain and an intracellular signaling domain comprising two functional signaling domains derived from one or more co-stimulatory molecule(s) and a functional signaling domain derived from a stimulatory molecule. The CAR can also comprise a chimeric fusion protein comprising an extracellular antigen recognition domain, a transmembrane domain and an intracellular signaling domain comprising at least two functional signaling domains derived from one or more co-stimulatory molecule(s) and a functional signaling domain derived from a stimulatory molecule. The antigen recognition moiety of the CAR encoded by the nucleic acid sequence can contain any lineage specific, antigen-binding antibody fragment. The antibody fragment can comprise one or more CDRs, the variable region (or portions thereof), the constant region (or portions thereof), or combinations of any of the foregoing.

The term “signaling domain” refers to the functional portion of a protein which acts by transmitting information within the cell to regulate cellular activity via defined signaling pathways by generating second messengers or functioning as effectors by responding to such messengers.

The term “zeta” or alternatively “zeta chain”, “CD3-zeta” or “TCR-zeta” is defined as the protein provided as GenBank accession numbers NP_932170, NP_000725, or XP_011508447; or the equivalent residues from a non-human species, e.g., mouse, rodent, monkey, ape and the like, and a “zeta stimulatory domain” or alternatively a “CD3-zeta stimulatory domain” or a “TCR-zeta stimulatory domain” is defined as the amino acid residues from the cytoplasmic domain of the zeta chain that are sufficient to functionally transmit an initial signal necessary for T cell activation.

The phrases “have antigen specificity” and “elicit antigen-specific response” as used herein means that the CAR can specifically bind to and immunologically recognize an antigen, such that binding of the CAR to the antigen elicits an immune response.

The extracellular antigen binding domain may be any protein or portion thereof that binds to a target protein, e.g., a receptor or ligand-binding portion thereof; a ligand of a receptor (e.g., a cytokine); or an antibody or antigen-binding portion of an antibody, e.g., a single-chain antibody (scFv).

In certain embodiments, a CAR comprises a transmembrane domain selected from the group consisting of a CD4 transmembrane domain, a CD8 transmembrane domain, and a CD28 transmembrane domain.

In certain embodiments, the intracellular signaling domain comprises a primary signaling domain, e.g., a T cell receptor zeta chain or primary signaling domain therefrom. In certain embodiments, the intracellular signaling domain further comprises one or more co-stimulatory domains. Illustrative examples of co-stimulatory domains that may be used in the CARs may include, but are not limited to: e.g., CD27, CD28, CD137 (4-1BB), OX-40, or combinations of thereof.

The CAR may be a first-generation, second-generation, or third-generation CAR. In particular embodiments, the CAR is encoded by an expression vector. The vector may be bicistronic, in particular embodiments. In some embodiments, more than one CAR is expressed by the immune cell. In particular embodiments where more than one CAR is to be expressed by the immune cell, the two or more CAR expression constructs may or may not be on the same vector. When present on the same vector, the first CAR coding sequence may be configured 5′ or 3′ to the second CAR coding sequence. The expression of the first CAR and second or subsequent CAR receptor may be under the direction of the same or different regulatory sequences.

### Suicide Genes

“Suicide genes” and “suicide gene systems” as described herein, can refer to methods to destroy a cell through apoptosis, which requires a suicide gene that will cause a cell to kill itself by apoptosis.

In certain embodiments, the suicide gene encodes one or more caspases. The suicide gene may encode Caspase 3, Caspase 7, Caspase 8 or Caspase 9.

In certain embodiments, the expression of certain gene products kills the immune cells under controlled conditions, such as inducible suicide genes.

### Bispecific Antibodies

The present cell/composition may be used in combination with a bispecific antibody or a diabody.

The term “bispecific antibody” or “multispecific antibody” refers to an antibody that recognized more than one epitope. Such antibodies are useful for targeting different antigens using the same antibody. Methods of making such antibodies are well-known in the art (see, at least U.S. Pat. No. 5,798,229; U.S. Pat. No. 5,989,830; and Holliger et al. (2005) Nat. Biotech. 23:1126-1136).

A “bispecific antibody” comprises two antigen binding sites having different antigen specificities.

As used herein, the term “diabodies” refers to small antibody fragments with two antigen-binding sites, which fragments comprise a heavy chain variable domain (V_{H}) connected to a light chain variable domain (V_{L}) in the same polypeptide chain. By using a linker that is too short to allow pairing between the two domains on the same chain, the domains are forced to pair with the complementary domains of another chain and create two antigen-binding sites. Diabodies are described more fully in Holliger et al. (1993) Proc. Natl. Acad. Sci. USA 90: 6444-6448 and Holliger and Hudson (2005) Nat. Biotechnol. 23:1126-1136.

### Transplantation

The present cells/compositions and methods can be administered to reduce the likelihood of, or increase the duration prior to, transplant rejection, or to induce immunosuppression.

The present cells/compositions and methods may be used in any situation in which immunosuppression is desired (e.g., transplant rejection or autoimmune disorders). The present cells/compositions and methods may be used to treat transplant rejection, e.g., reducing the likelihood that a particular transplant is rejected by the host or increasing the time before rejection takes place. The present cells/compositions and methods may can be used in conjunction with transplantation of any organ or any tissue that is suitable for transplantation. Non-limiting exemplary organs include heart, kidney, lung, liver, pancreas, intestine, and thymus; non-limiting exemplary tissues include bone, tendon, cornea, skin, heart valve, vein, and bone marrow.

The present disclosure provides for a method of inducing immune tolerance, or treating or preventing rejection, for xenotransplantation in a subject to a graft obtained from a donor mammal. The method may comprise administering the present cell/composition to the subject before, during or after transplantation.

“Xenogeneic” refers to deriving from, originating in, or being members of different species, e.g., human and swine, human and chimpanzee, human and rodent, etc. A “xenogeneic transplantation” or “xenotransplantation” refers to transfer of cells, tissues or organs from a donor to a recipient where the recipient is a species different from that of the donor.

The second species may be swine, such as a miniature swine.

The first species is may be primate, such as non-human primate or human.

The graft may comprise cells, a tissue or an organ. In one embodiment, the graft comprises hematopoietic stem cells. In another embodiment, the graft comprises bone marrow. In yet another embodiment, the graft comprises a heart, a kidney, a liver, a pancreas, a lung, an intestine, skin, a small bowel, a trachea, a cornea, or combinations thereof.

“Tolerance”, as used herein, refers to the inhibition or decrease of a graft recipient's ability to mount an immune response, e.g., to a donor antigen, which would otherwise occur, e.g., in response to the introduction of a non self MHC antigen into the recipient. Tolerance can involve humoral, cellular, or both humoral and cellular responses. The concept of tolerance includes both complete and partial tolerance. In other words, as used herein, tolerance include any degree of inhibition of a graft recipient's ability to mount an immune response, e.g., to a donor antigen.

“Miniature swine”, as used herein, refers to completely or partially inbred miniature swine.

“Graft”, as used herein, refers to a body part, organ, tissue, cells, or portions thereof.

Restoring, inducing, or promoting immunocompetence, as used herein, means one or both of: (1) increasing the number of mature functional T cells in the recipient (over what would be seen in the absence of treatment with a method of the invention) by either or both, increasing the number of recipient-mature functional T cells or by providing mature functional donor-T cells, which have matured in the recipient; or (2) improving the immune-responsiveness of the recipient, e.g., as is measured by the ability to mount a skin response to a recall antigen, or improving the responsiveness of a T cell of the recipient, e.g., as measured by an in vitro test, e.g., by the improvement of a proliferative response to an antigen.

In certain embodiments, preparation of the recipient for either organ transplantation or thymus replacement includes any or all of the following steps. They may be carried out in the following sequence.

First, a preparation of horse anti-human thymocyte globulin (ATG) is intravenously injected into the recipient. The antibody preparation eliminates mature T cells and natural killer cells. If not eliminated, mature T cells might promote rejection of both the thymic transplant and, after sensitization, the xenograft organ. The ATG preparation also eliminates natural killer (NK) cells. NK cells probably have no effect on an implanted organ, but might act immediately to reject the newly introduced thymic tissue. Anti-human ATG obtained from any mammalian host can also be used, e.g., ATG produced in pigs, although thus far preparations of pig ATG have been of lower titer than horse-derived ATG. ATG is superior to anti-NK monoclonal antibodies, as the latter are generally not lytic to all host NK cells, while the polyclonal mixture in ATG is capable of lysing all host NK cells. Anti-NK monoclonal antibodies can, however, be used. In a relatively severely immunocompromised individual this step may not be necessary. As host (or donor) T cells mature in the xenogeneic thymus they will be tolerant of the thymic tissue. Alternatively, as the host immune system is progressively restored, it may be desirable to treat the host to induce tolerance to the thymic tissue.

Optimally, the recipient can be thymectomized. In thymectomized recipients, recipient T cells do not have an opportunity to differentiate in the recipient thymus, but must differentiate in the hybrid thymic tissue. In some cases, it may be necessary to splenectomize the recipient in order to avoid anemia.

Second, the recipient can be administered low dose radiation. Although this step is thought to be beneficial in bone marrow transplantation (by creating hematopoietic space for newly injected bone marrow cells), it is of less importance in thymic grafts which are not accompanied by bone marrow transplantation. However, a sublethal dose e.g., a dose about equal to 100, or more than 100 and less than about 400, rads, whole body radiation, plus 700 rads of local thymic radiation, can be used.

Third, natural antibodies can be adsorbed from the recipient's blood. Antibody removal can be accomplished by exposing the recipient's blood to donor or donor species antigens, e.g., by hemoperfusion of a liver of the donor species to adsorb recipient-natural antibodies. Pre-formed natural antibodies (nAb) are the primary agents of graft rejection. Natural antibodies bind to xenogeneic endothelial cells and are primarily of the IgM class. These antibodies are independent of any known previous exposure to antigens of the xenogeneic donor. B cells that produce these natural antibodies tend to be T cell-independent, and are normally tolerized to self antigen by exposure to these antigens during development. Again, this step may not be required, at least initially, in a relatively severely immunocompromised patient.

The hybrid thymic tissue is implanted in the recipient. Fetal or neonatal liver or spleen tissue can be included.

One, or any combination including all, of these procedures may aid the survival of implanted thymic tissue or another xenogeneic organ.

Methods of the present disclosure can be used to confer tolerance to xenogeneic grafts, e.g., wherein the graft donor is a nonhuman animal, e.g., a swine, e.g., a miniature swine, and the graft recipient is a primate, e.g., a human.

The donor of the xenograft and the individual that supplies the tolerance-inducing thymic tissue may be the same individual or may be as closely related as possible. For example, it is preferable to derive a xenograft from a colony of donors that is highly or completely inbred.

The donor may be a non-human mammalian species, such as a swine (e.g., a miniature swine) or a non-human primate. Non-limiting examples of the donor include a swine, rodent, non-human primate, cow, goat, and horse.

In one embodiment, the donor is a miniature swine which is at least partially inbred (e.g., the swine is homozygous at swine leukocyte antigen (SLA) loci, and/or is homozygous at at least 65%, 70%, 75%, 80%, 85%, 90%, 95%, or more, of all other genetic loci. The genetic engineering can be made in wholly or partially inbred swine (e.g., miniature swine, transgenic swine, etc.). For example, inbred Massachusetts General Hospital (MGH) miniature swine may be used in the present methods. These include the MGH miniature swine which have been inbred for over 40 years and are homozygous at all genetic loci. In one embodiment, inbred SLA^{dd} miniature swine may be used. Mezrich et al. and Sachs, Histocompatible miniature swine: An inbred large-animal model. Transplantation, 2003; 75:904-907. Swine at National Swine Resource and Research Center (NSRRC, RADIL, University Missouri, Columbia Mo.) may also be used in the present methods.

The recipient may be a primate, such as non-human primate (e.g., a baboon, or cynomolgus monkey) or human. In one embodiment, the recipient is human.

In certain embodiments, the donor and recipient are of different species. For example, the donor is a non-human animal, e.g., a miniature swine, and the recipient is a human.

Also encompassed by the present disclosure are methods of transplanting a graft from such a donor animal into a recipient (e.g., human).

Cells, tissues, organs or body fluids of the present donor animal may be used for transplantation (e.g., xenotransplantation). The graft harvested from the donor for transplantation may include, but are not limited to, a heart, a kidney, a liver, a pancreas, a lung transplant, an intestine, skin, thyroid, bone marrow, small bowel, a trachea, a cornea, a limb, a bone, an endocrine gland, blood vessels, connective tissue, progenitor stem cells, blood cells, hematopoietic cells, Islets of Langerhans, brain cells and cells from endocrine and other organs, bodily fluids, and combinations thereof.

The cell can be any type of cell. In certain embodiments, the cell is a hematopoietic cell (e.g., a hematopoietic stem cell, lymphocyte, a myeloid cell), a pancreatic cell (e.g., a beta-islet cell), a kidney cell, a heart cell, or a liver cell.

Bone marrow cells, or hematopoietic stem cells (e.g., a fetal liver suspension or mobilized peripheral blood stem cells) of the donor animal may be injected into the recipient.

Treatments that promote tolerance and/or decrease immune recognition of the graft may also include use of immunosuppressive agents (e.g., cyclosporine, FK506), antibodies (e.g., anti-T cell antibodies such as polyclonal anti-thymocyte antisera (ATG), and/or a monoclonal anti-human T cell antibody, such as LoCD2b), irradiation, and methods to induce mixed chimerism. U.S. Pat. Nos. 6,911,220; 6,306,651; 6,412,492; 6,514,513; 6,558,663; and 6,296,846. Kuwaki et al., Nature Med., 11(1):29-31, 2005. Yamada et al., Nature Med. 11 (1):32-34, 2005.

In some embodiments, the recipient is thymectomized and/or splenectomized. Thymic irradiation can be used.

In some embodiments, the recipient is administered low dose radiation (e.g., a sublethal dose of between 100 rads and 400 rads whole body radiation). Local thymic radiation may also be used.

The recipient can be treated with an agent that depletes complement, such as cobra venom factor.

Natural antibodies of the recipient may be eliminated by organ perfusion, and/or transplantation of tolerance-inducing bone marrow. Natural antibodies can be absorbed from the recipient's blood by hemoperfusion of a liver of the donor species. The cells, tissues, or organs used for transplantation may be genetically modified such that they are not recognized by natural antibodies of the host (e.g., the cells are a-1,3-galactosyltransferase deficient).

In some embodiments, the methods include treatment with a human anti-human CD154 mAb, mycophenolate mofetil, and/or methylprednisolone. The methods can also include agents useful for supportive therapy such as anti-inflammatory agents (e.g., prostacyclin, dopamine, ganiclovir, levofloxacin, cimetidine, heparin, antithrombin, erythropoietin, and aspirin).

In some embodiments, donor stromal tissue is administered.

An immunosuppressant, also referred to as an immunosuppressive agent, can be any compound that decreases the function or activity of one or more aspects of the immune system, such as a component of the humoral or cellular immune system or the complement system.

Non-limiting examples of immunosuppressants include, (1) antimetabolites, such as purine synthesis inhibitors (such as inosine monophosphate dehydrogenase inhibitors, e.g., azathioprine, mycophenolate, and mycophenolate mofetil), pyrimidine synthesis inhibitors (e.g., leflunomide and teriflunomide), and antifolates (e.g., methotrexate); (2) calcineurin inhibitors, such as tacrolimus, cyclosporine A, pimecrolimus, and voclosporin; (3) TNF-alpha inhibitors, such as thalidomide and lenalidomide; (4) IL-1 receptor antagonists, such as anakinra; (5) mammalian target of rapamycin (mTOR) inhibitors, such as rapamycin (sirolimus), deforolimus, everolimus, temsirolimus, zotarolimus, and biolimus; (6) corticosteroids, such as prednisone; and (7) antibodies to any one of a number of cellular or serum targets (including anti-lymphocyte globulin and anti-thymocyte globulin).

In one embodiment, a recipient is treated with a preparation of horse anti-human thymocyte globulin (ATG) injected intravenously (e.g., at a dose of approx. 25-100 mg/kg, e.g., 50 mg/kg, e.g., at days-3, -2, -1 prior to transplantation). The antibody preparation eliminates mature T cells and natural killer (NK) cells. The ATG preparation also eliminates NK cells. Anti-human ATG obtained from any mammalian host can also be used. In addition, if further T cell depletion is indicated, the recipient may be treated with a monoclonal anti-human T cell antibody, such as LoCD2b (Immerge BioTherapeutics, Inc., Cambridge, Mass.). For bone marrow transplant, the recipient can be administered low dose radiation. In some cases, the recipient can be treated with an agent that depletes complements, such as cobra venom factor (e.g., at day-1).

In some embodiments, maintenance therapy (e.g., beginning immediately prior to, and continuing for at least a few days after transplantation) includes treatment with a human anti-human CD154 mAb. Mycophenolate mofetil may be administered to maintain the whole blood levels. Methylprednisolone may also be administered, beginning on the day of transplantation, tapering thereafter over the next 3-4 weeks.

Various agents useful for supportive therapy (e.g., at days 0-14) include anti-inflammatory agents such as prostacyclin, dopamine, ganiclovir, levofloxacin, cimetidine, heparin, antithrombin, erythropoietin, and aspirin.

In some embodiments, donor stromal tissue is administered. It may be obtained from fetal liver, thymus, and/or fetal spleen, may be implanted into the recipient, e.g., in the kidney capsule. Thymic tissue can be prepared for transplantation by implantation under the autologous kidney capsule for revascularization. Stem cell engraftment and hematopoiesis across disparate species barriers may be enhanced by providing a hematopoietic stromal environment from the donor species. The stromal matrix supplies species-specific factors that are required for interactions between hematopoietic cells and their stromal environment, such as hematopoietic growth factors, adhesion molecules, and their ligands.

As liver is the major site of hematopoiesis in the fetus, fetal liver can also serve as an alternative to bone marrow as a source of hematopoietic stem cells. Each organ includes an organ specific stromal matrix that can support differentiation of the respective undifferentiated stem cells implanted into the host. As an alternative or an adjunct to implantation, fetal liver cells can be administered in fluid suspension.

Bone marrow cells, or another source of hematopoietic stem cells, e.g., a fetal liver suspension, of the donor can be injected into the recipient. Donor bone marrow cells home to appropriate sites of the recipient and grow contiguously with remaining host cells and proliferate, forming a chimeric lymphohematopoietic population. By this process, newly forming B cells (and the antibodies they produce) are exposed to donor antigens, so that the transplant will be recognized as self. Tolerance to the donor is also observed at the T cell level in animals in which hematopoietic stem cell, e.g., bone marrow cells, engraftment has been achieved. The use of xenogeneic donors allows the possibility of using bone marrow cells and organs from the same animal, or from genetically matched animals.

### Autoimmune Disorders

The present cells/compositions and methods may have in vitro and in vivo therapeutic, prophylactic, and/or diagnostic utilities.

The present cells/compositions and methods may treat or prevent an autoimmune disorder.

The autoimmune disorder may be associated with or caused by the presence of an autoantibody.

In some embodiments, the autoimmune disorder is Type 1 diabetes.

The autoimmune disorder may be systemic lupus erythematosus (SLE), CREST syndrome (calcinosis, Raynaud's syndrome, esophageal dysmotility, sclerodactyl, and telangiectasia), opsoclonus, inflammatory myopathy (e.g., polymyositis, dermatomyositis, and inclusion-body myositis), systemic scleroderma, primary biliary cirrhosis, celiac disease (e.g., gluten sensitive enteropathy), dermatitis herpetiformis, Miller-Fisher Syndrome, acute motor axonal neuropathy (AMAN), multifocal motor neuropathy with conduction block, autoimmune hepatitis, antiphospholipid syndrome, Wegener's granulomatosis, microscopic polyangiitis, Churg-Strauss syndrome, rheumatoid arthritis, chronic autoimmune hepatitis, scleromyositis, myasthenia gravis, Lambert-Eaton myasthenic syndrome, Hashimoto's thyroiditis, Graves' disease, Paraneoplastic cerebellar degeneration, Stiff person syndrome, limbic encephalitis, Isaacs Syndrome, Sydenham's chorea, pediatric autoimmune neuropsychiatric disease associated with Streptococcus (PANDAS), encephalitis, diabetes mellitus type 1, and/or Neuromyelitis optica.

The autoimmune disorder may be pernicious anemia, Addison's disease, psoriasis, inflammatory bowel disease, psoriatic arthritis, Sjogren's syndrome, lupus erythematosus (e.g., discoid lupus erythematosus, drug-induced lupus erythematosus, and neonatal lupus erythematosus), multiple sclerosis, and/or reactive arthritis.

The autoimmune disorder may be polymyositis, dermatomyositis, multiple endocrine failure, Schmidt's syndrome, autoimmune uveitis, adrenalitis, thyroiditis, autoimmune thyroid disease, gastric atrophy, chronic hepatitis, lupoid hepatitis, atherosclerosis, presenile dementia, demyelinating diseases, subacute cutaneous lupus erythematosus, hypoparathyroidism, Dressler's syndrome, autoimmune thrombocytopenia, idiopathic thrombocytopenic purpura, hemolytic anemia, pemphigus vulgaris, pemphigus, alopecia arcata, pemphigoid, scleroderma, progressive systemic sclerosis, adult onset diabetes mellitus (e.g., type II diabetes), male and female autoimmune infertility, ankylosing spondolytis, ulcerative colitis, Crohn's disease, mixed connective tissue disease, polyarteritis nedosa, systemic necrotizing vasculitis, juvenile onset rheumatoid arthritis, glomerulonephritis, atopic dermatitis, atopic rhinitis, Goodpasture's syndrome, Chagas' disease, sarcoidosis, rheumatic fever, asthma, recurrent abortion, anti-phospholipid syndrome, farmer's lung, erythema multiforme, post cardiotomy syndrome, Cushing's syndrome, autoimmune chronic active hepatitis, bird-fancier's lung, allergic disease, allergic encephalomyelitis, toxic epidermal necrolysis, alopecia, Alport's syndrome, alveolitis, allergic alveolitis, fibrosing alveolitis, interstitial lung disease, erythema nodosum, pyoderma gangrenosum, transfusion reaction, leprosy, malaria, leishmaniasis, trypanosomiasis, Takayasu's arteritis, polymyalgia rheumatica, temporal arteritis, schistosomiasis, giant cell arteritis, ascariasis, aspergillosis, Sampter's syndrome, eczema, lymphomatoid granulomatosis, Behcet's disease, Caplan's syndrome, Kawasaki's disease, dengue, endocarditis, endomyocardial fibrosis, endophthalmitis, erythema elevatum et diutinum, erythroblastosis fetalis, eosinophilic faciitis, Shulman's syndrome, Felty's syndrome, filariasis, cyclitis, chronic cyclitis, heterochronic cyclitis, Fuch's cyclitis, IgA nephropathy, Henoch-Schonlein purpura, graft versus host disease, transplantation rejection, human immunodeficiency virus infection, echovirus infection, cardiomyopathy, Alzheimer's disease, parvovirus infection, rubella virus infection, post vaccination syndromes, congenital rubella infection, Hodgkin's and non-Hodgkin's lymphoma, renal cell carcinoma, multiple myeloma, Eaton-Lambert syndrome, relapsing polychondritis, malignant melanoma, cryoglobulinemia, Waldenstrom's macroglobulemia, Epstein-Barr virus infection, mumps, Evan's syndrome, and/or autoimmune gonadal failure.

Autoimmune diseases that may be treated with the present cells/compositions and methods may include, but are not limited to, systemic lupus erythematosus (SLE), CREST syndrome (calcinosis, Raynaud's syndrome, esophageal dysmotility, sclerodactyl, and telangiectasia), opsoclonus, inflammatory myopathy (e.g., polymyositis, dermatomyositis, and inclusion-body myositis), systemic scleroderma, primary biliary cirrhosis, celiac disease (e.g., gluten sensitive enteropathy), dermatitis herpetiformis, Miller-Fisher Syndrome, acute motor axonal neuropathy (AMAN), multifocal motor neuropathy with conduction block, autoimmune hepatitis, antiphospholipid syndrome, Wegener's granulomatosis, microscopic polyangiitis, Churg-Strauss syndrome, rheumatoid arthritis, chronic autoimmune hepatitis, scleromyositis, myasthenia gravis, Lambert-Eaton myasthenic syndrome, Hashimoto's thyroiditis, Graves' disease, Paraneoplastic cerebellar degeneration, Stiff person syndrome, limbic encephalitis, Isaacs Syndrome, Sydenham's chorea, pediatric autoimmune neuropsychiatric disease associated with Streptococcus (PANDAS), encephalitis, diabetes mellitus type 1, and Neuromyelitis optica.

Other autoimmune disorders include pernicious anemia, Addison's disease, psoriasis, inflammatory bowel disease, psoriatic arthritis, Sjogren's syndrome, lupus erythematosus (e.g., discoid lupus erythematosus, drug-induced lupus erythematosus, and neonatal lupus erythematosus), multiple sclerosis, and reactive arthritis.

Additional disorders that may be treated using the cells/compositions and methods of the present disclosure include, for example, polymyositis, dermatomyositis, multiple endocrine failure, Schmidt's syndrome, autoimmune uveitis, adrenalitis, thyroiditis, autoimmune thyroid disease, gastric atrophy, chronic hepatitis, lupoid hepatitis, atherosclerosis, presenile dementia, demyelinating diseases, subacute cutaneous lupus erythematosus, hypoparathyroidism, Dressler's syndrome, autoimmune thrombocytopenia, idiopathic thrombocytopenic purpura, hemolytic anemia, pemphigus vulgaris, pemphigus, alopecia arcata, pemphigoid, scleroderma, progressive systemic sclerosis, adult onset diabetes mellitus (e.g., type II diabetes), male and female autoimmune infertility, ankylosing spondolytis, ulcerative colitis, Crohn's disease, mixed connective tissue disease, polyarteritis nedosa, systemic necrotizing vasculitis, juvenile onset rheumatoid arthritis, glomerulonephritis, atopic dermatitis, atopic rhinitis, Goodpasture's syndrome, Chagas' disease, sarcoidosis, rheumatic fever, asthma, recurrent abortion, anti-phospholipid syndrome, farmer's lung, erythema multiforme, post cardiotomy syndrome, Cushing's syndrome, autoimmune chronic active hepatitis, bird-fancier's lung, allergic disease, allergic encephalomyelitis, toxic epidermal necrolysis, alopecia, Alport's syndrome, alveolitis, allergic alveolitis, fibrosing alveolitis, interstitial lung disease, erythema nodosum, pyoderma gangrenosum, transfusion reaction, leprosy, malaria, leishmaniasis, trypanosomiasis, Takayasu's arteritis, polymyalgia rheumatica, temporal arteritis, schistosomiasis, giant cell arteritis, ascariasis, aspergillosis, Sampter's syndrome, eczema, lymphomatoid granulomatosis, Behcet's disease, Caplan's syndrome, Kawasaki's disease, dengue, endocarditis, endomyocardial fibrosis, endophthalmitis, erythema elevatum et diutinum, erythroblastosis fetalis, eosinophilic faciitis, Shulman's syndrome, Felty's syndrome, filariasis, cyclitis, chronic cyclitis, heterochronic cyclitis, Fuch's cyclitis, IgA nephropathy, Henoch-Schonlein purpura, graft versus host disease, transplantation rejection, human immunodeficiency virus infection, echovirus infection, cardiomyopathy, Alzheimer's disease, parvovirus infection, rubella virus infection, post vaccination syndromes, congenital rubella infection, Hodgkin's and non-Hodgkin's lymphoma, renal cell carcinoma, multiple myeloma, Eaton-Lambert syndrome, relapsing polychondritis, malignant melanoma, cryoglobulinemia, Waldenstrom's macroglobulemia, Epstein-Barr virus infection, mumps, Evan's syndrome, and autoimmune gonadal failure.

### Pharmaceutical Compositions

The present disclosure provides pharmaceutical compositions comprising the present cells. The cells or pharmaceutical compositions of the present invention may be administered by any route, including, without limitation, oral, transdermal, ocular, intraperitoneal, intravenous, ICV, intracisternal injection or infusion, subcutaneous, implant, sublingual, subcutaneous, intramuscular, intravenous, rectal, mucosal, ophthalmic, intrathecal, intra-articular, intra-arterial, sub-arachinoid, bronchial and lymphatic administration. The present composition may be administered parenterally or systemically.

Intravenous forms include, but are not limited to, bolus and drip injections. Examples of intravenous dosage forms include, but are not limited to, Water for Injection USP; aqueous vehicles including, but not limited to, Sodium Chloride Injection, Ringer's Injection, Dextrose Injection, Dextrose and Sodium Chloride Injection, and Lactated Ringer's Injection; water-miscible vehicles including, but not limited to, ethyl alcohol, polyethylene glycol and polypropylene glycol; and non-aqueous vehicles including, but not limited to, corn oil, cottonseed oil, peanut oil, sesame oil, ethyl oleate, isopropyl myristate and benzyl benzoate.

The present compound(s) or composition may be administered as a single dose, or as two or more doses (which may or may not contain the same amount of the desired molecule) over time, or as a continuous infusion via implantation device or catheter. The pharmaceutical composition can be prepared in single unit dosage forms.

Appropriate frequency of administration can be determined by one of skill in the art and can be administered once or several times per day (e.g., twice, three, four or five times daily). The compositions of the invention may also be administered once each day or once every other day. The compositions may also be given twice weekly, weekly, monthly, or semi-annually. In the case of acute administration, treatment is typically carried out for periods of hours or days, while chronic treatment can be carried out for weeks, months, or even years. U.S. Pat. No. 8,501,686.

Administration of the compositions of the invention can be carried out using any of several standard methods including, but not limited to, continuous infusion, bolus injection, intermittent infusion, or combinations of these methods. For example, one mode of administration that can be used involves continuous intravenous infusion. The infusion of the compositions of the invention can, if desired, be preceded by a bolus injection.

As used herein, the term “therapeutically effective amount” is an amount sufficient to treat a specified disorder or disease or alternatively to obtain a pharmacological response treating a disorder or disease.

Methods of determining the most effective means and dosage of administration can vary with the composition used for therapy, the purpose of the therapy, the target cell being treated, and the subject or patient being treated. Single or multiple administrations can be carried out with the dose level and pattern being selected by the treating physician. The specific dose level for any particular subject depends upon a variety of factors including the age, body weight, general health, sex, diet, time of administration, route of administration, and rate of excretion, and the severity of the particular disease undergoing therapy.

Tregs may be administered at 0.1×10⁶, 0.2×10⁶, 0.3×10⁶, 0.4×10⁶, 0.5×10⁶, 0.6×10⁶, 0.7×10⁶, 0.8×10⁶, 0.9×10⁶, 1.0×10⁶, 5.0×10⁶, 1.0×10⁷, 5.0×10⁷, 1.0×10⁸, 5.0×10⁸, or more, or any range in between or any value in between, cells per kilogram of subject body weight. The number of cells administered may be adjusted. Generally, 1×10⁵ to about 1×10⁹ cells/kg of body weight, from about 1×10⁶ to about 1×10⁸ cells/kg of body weight, or about 1×10⁷ cells/kg of body weight, or more cells, as necessary, may be administered.

Different dosage regimens may be used. In some embodiments, a daily dosage, such as any of the exemplary dosages described above, is administered once, twice, three times, or four times a day for at least three, four, five, six, seven, eight, nine, or ten days. Depending on the stage and severity of the cancer, a shorter treatment time (e.g., up to five days) may be employed along with a high dosage, or a longer treatment time (e.g., ten or more days, or weeks, or a month, or longer) may be employed along with a low dosage. In some embodiments, a once- or twice-daily dosage is administered every other day.

Administration can be accomplished using methods generally known in the art. The cells/composition may be administered to the desired site by direct injection, or by any other means used in the art including, but are not limited to, intravascular, intracerebral, parenteral, intraperitoneal, intravenous, epidural, intraspinal, intrasternal, intra-articular, intra-synovial, intrathecal, intra-arterial, intracardiac, or intramuscular administration. For example, subjects of interest may be administered with the cells/composition by various routes. Such routes include, but are not limited to, intravenous administration, subcutaneous administration, administration to a specific tissue (e.g., focal transplantation), injection into the femur bone marrow cavity, injection into the spleen, administration under the renal capsule of fetal liver, and the like. Cells may be administered in one infusion, or through successive infusions over a defined time period sufficient to generate a desired effect.

Administration of a therapeutically active amount of the present cells/composition may be defined as an amount effective, at dosages and for periods of time necessary, to achieve the desired result. For example, a therapeutically active amount of the present cells/composition may vary according to factors such as the disease state, age, sex, and weight of the individual, and the ability of peptide to elicit a desired response in the individual. Dosage regimens can be adjusted to provide the optimum therapeutic response. For example, several divided doses can be administered daily or the dose can be proportionally reduced as indicated by the exigencies of the therapeutic situation.

### Kits

The present disclosure provides for a kit for use in the treatment or prevention of an autoimmune disorder. The present disclosure also provides for a kit for use in inducing immune tolerance, or treating or preventing rejection, for xenotransplantation.

Kits according to the present disclosure include package(s) (e.g., vessels) comprising the present cell or compositions. The cells may be present in the pharmaceutical compositions as described herein. The cells may be present in unit dosage forms.

Examples of pharmaceutical packaging materials include, but are not limited to, bottles, tubes, inhalers, pumps, bags, vials, containers, syringes, bottles, and any packaging material suitable for a selected formulation and intended mode of administration and treatment.

Kits can contain instructions for administering the present cells or compositions to a patient. Kits also can comprise instructions for uses of the present cells or compositions. Kits also can contain labeling or product inserts for the cells/compositions. The kits also can include buffers for preparing solutions for conducting the methods.

### Definitions

The terms “subject,” “individual,” and “patient” are used interchangeably, and refer to a vertebrate, preferably a mammal such as a human. Mammals include, but are not limited to, human primates, non-human primates or murine, bovine, equine, canine or feline species. In the context of the present disclosure, the term “subject” also encompasses tissues and cells that can be cultured in vitro or ex vivo or manipulated in vivo. The term “subject” can be used interchangeably with the term “organism”.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Examples of polynucleotides include, but are not limited to, coding or non-coding regions of a gene or gene fragment, messenger RNA (mRNA), cDNA, recombinant polynucleotides, branched polynucleotides, plasmids, vectors, isolated DNA of any sequence, isolated RNA of any sequence, nucleic acid probes, and primers. One or more nucleotides within a polynucleotide can further be modified. The sequence of nucleotides may be interrupted by non-nucleotide components.

The term “genetically engineered” or “genetically modified” refers to cells being manipulated by genetic engineering, for example by genome editing. That is, the cells contain a heterologous sequence which does not naturally occur in said cells. The heterologous nucleic acid molecule may be integrated into the genome of the cells or may be present extra-chromosomally, e.g., in the form of plasmids. The term also includes embodiments of introducing genetically engineered, isolated CAR polypeptides into the cell.

As used herein, the terms “under the control”, “under transcriptional control”, “operably positioned”, and “operably linked” mean that a promoter is in a correct functional location and/or orientation in relation to a nucleic acid sequence, a DNA fragment, or a gene, to control transcriptional initiation and/or expression of that sequence, DNA fragment or gene.

The term “autologous” refers to any material derived from the same individual to whom it is later to be re-introduced into the same individual.

The term “allogeneic” refers to any material derived from a different animal of the same species as the individual to whom the material is introduced. Two or more individuals are said to be allogeneic to one another when the genes at one or more loci are not identical.

The following are examples of the present invention and are not to be construed as limiting.

### EXAMPLES

### Example 1

### Generation of Conversion/Conditioning-Resistant CAR Tregs for Delivery of Immunomodulatory Molecules in Order to Locally Suppress the Innate and Adoptive Immunity in Transplantation and Autoimmunity Settings

Despite promising results in using CAR Tregs for tolerance induction, there are still major considerations regarding the plasticity of CAR Tregs (Koenen et al. 2008) and the effect of T cell-depleting conditioning regimens (Neelapu 2019; Mancusi et al. 2019) on these cells that need to be addressed before their clinical application Inflammation is shown to drive Treg conversion (Hua et al 2018). CAR Tregs can quickly reject the target organs if they lose their regulatory phenotype and convert to effector T cells (Teff). We will use genetic engineering approaches to induce apoptotic cell death and/or block the CAR expression in CAR Tregs that convert to Teff. We will use the HLA-A2 CAR Treg to control autoimmune responses against HLA-A2+ human islets in a humanized mouse model of type 1 diabetes (T1D).

In recent years, a new generation of CAR T cells, named T cells redirected for universal cytokine killing (TRUCKs), has been developed, which is engineered to express certain cytokines upon encountering a tumor antigen, driven by a nuclear factor of activated T cells (NFAT)-responsive promoter (reviewed in Tian et al. 2020).

In order to improve the functionality of CAR Tregs in suppressing local innate and adoptive immune responses, we will use a similar system in CAR Tregs, in which the NFAT-responsive promoter drives the expression of immunomodulatory molecules targeting both adaptive and innate immune responses. This system not only allows us to enhance the capability of CAR Tregs to control innate and adaptive immune responses, but also gives us the opportunity to deliver other therapeutic factors.

A) Engineering CAR Tregs to prevent conversion to Teff. We will use two engineering strategies to prevent conversion of CAR Tregs to Teff cells. These two strategies will be tested both in vitro and in vivo.

i) Design of CAR Tregs to Prevent Conversion to Teff

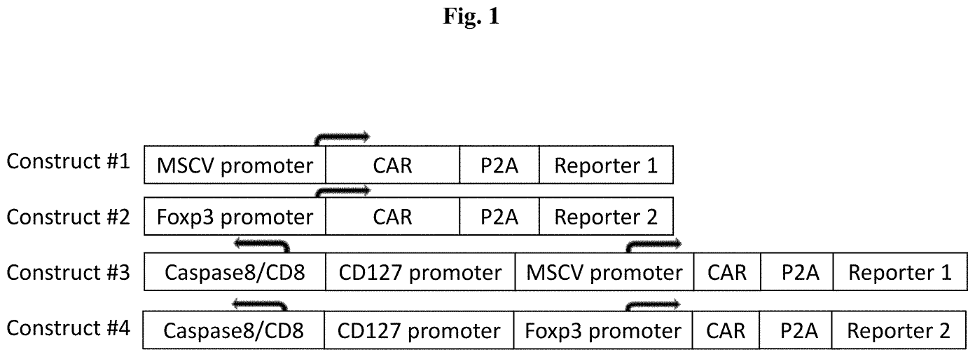

For the autoimmunity model, the HLA-A2 sequences having the single-chain variable fragments (scFv), followed by the CD28 and CD3-zeta sequences are designed under a ubiquitous promoter (MSCV) (Li et al. 2019) (construct #1, FIG. 1) or a Treg-specific promoter (Foxp3) (Mantel et al. 2006) (construct #2, FIG. 1). Introduction of the CARs under the Foxp3 promoter will ensure their silencing as soon as the Treg converts to a Foxp3-negative Teff, thereby preventing the Teff from killing the target cells. To provide another layer of protection, an inducible suicide gene is also be implemented. A chimeric CD8/caspase 8 sequence (Carlotti et al. 2005) is designed under the promoter of the IL-7 receptor a (CD127) (DeKoter et al. 2007), which inversely correlates with Foxp3 expression (Liu et al. 2006), to ensure that CAR Tregs undergo apoptosis as soon as they acquire an effector phenotype. The transmembrane domain of CD8 in this chimeric molecule allows for the oligomerization of caspase 8 in the membrane, which is an essential step in initiation of the cascade of events that leads to apoptosis (Martin et al. 1998). This sequence will be placed on opposite strands of the same vectors as constructs 1 and 2, to generate constructs #3 and #4, respectively (FIG. 1). The essential regions of both Foxp3 (Li et al. 2019) and CD127 (DeKoter et al. 2007) promoters have been identified. Therefore, we will use the minimal promoters found in these studies.

ii) In vitro models for evaluating CAR Treg suppressive capacity and conversion rate.

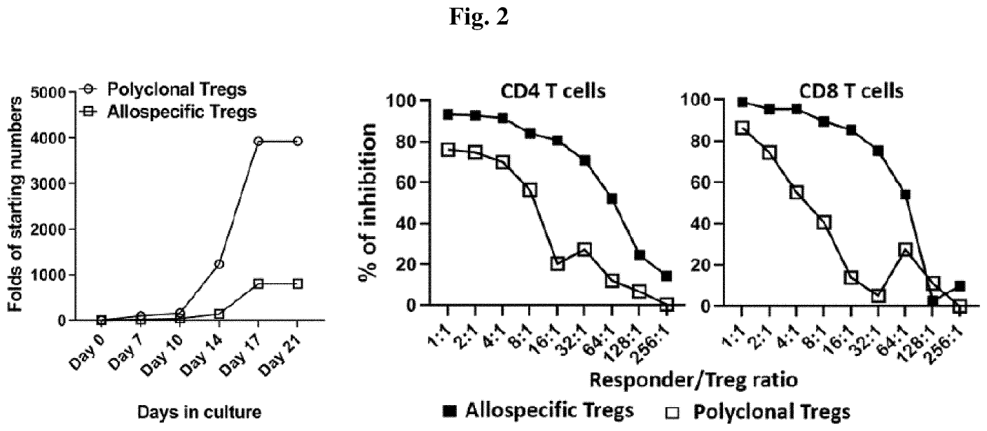

We will adjust our protocols in performing Treg suppression assay using humanized mouse Tregs (FIG. 2) in order to evaluate the capacity of HLA-A2 CAR Tregs in suppressing the proliferation of effector T cells.

Autoimmunity model: We will use 1E6 TCR-transduced T cells as responder cells in this model. The 1E6 TCR is a class I-restricted TCR, which kills human β cells through recognition of preproinsulin 15-24 (PPI:15-24) presented on HLA-A2 (Skowera et al. 2008; Bulek et al. 2012). We have generated a lentiviral vector containing this TCR. Human Tregs are sorted from an HLA-A2+ donor PBMC, activated and then transduced with the 4 different HLA-A2 CAR constructs. Autologous human T cells will be transduced with the 1E6 TCR (as the responders). Autologous PBMC-derived dendritic cells (DCs) loaded with PPI:15-24 will serve as the stimulator cells. Responders and CAR Tregs are stained with different proliferation dyes and the suppressive capacity and conversion rates of CAR Tregs at different groups in both culture conditions with and without conversion-forcing cytokines will be evaluated.

iii) In vivo models for evaluating CAR Treg suppressive capacity and conversion rate.

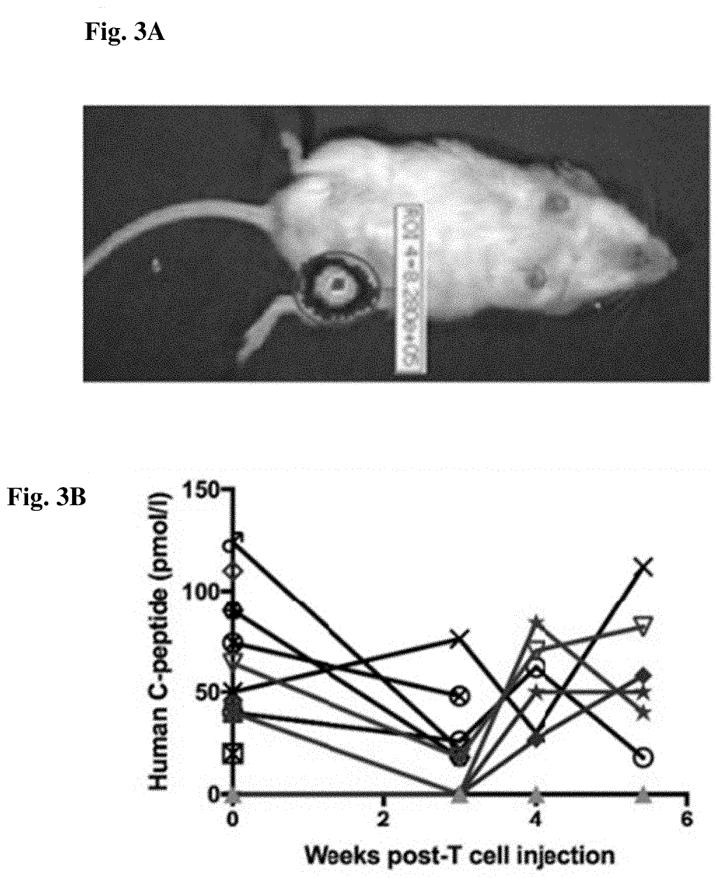

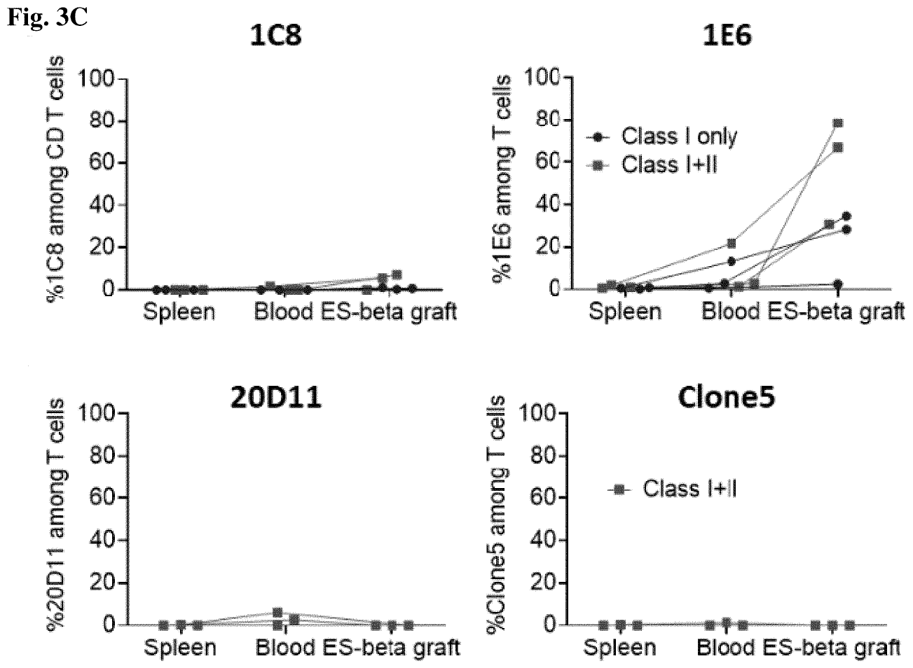

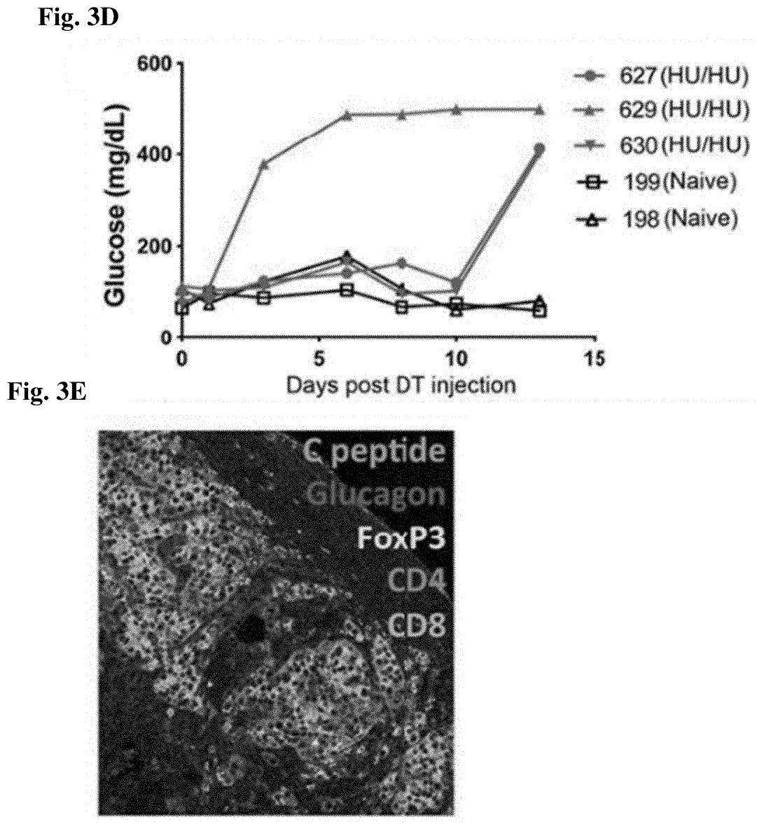

T1D model: In order to establish a T1D humanized mouse model, we transplanted human ES-derived β cells generated from the luciferase-expressing line into the muscle of humanized mice generated with HLA-A2+ fetal thymus and HSCs. As shown in FIG. 3A, transplantation of 3000 ES-P clusters (3M cells total) yields a reliable luciferase signal. The amount of C-peptide detected in these mice was detectable (FIG. 3B) but lower than what we typically obtain with human islets (500-1000 pmol/l for 2000 islet equivalents transplanted under the kidney capsule). Next, to explore the susceptibility of ES-β cells to autoimmune attack by T cells in our model, we collected T cells from the spleen of one mouse in the cohort and transduced them with 4 different insulin-reactive TCRs. Clone 5 and 20D11 TCRs recognize InsB:9-23 in the context of HLA-DQ8 (Class II). 1E6 and 1C8 recognize PPI:15-24 and PPI:2-12, respectively, both in the context of HLA-A2. 3 mice received T cells with the 2 class I TCRs (1E6 and 20D11) and the other 3 mice received T cells with both class I and class II TCRs. Potential killing of ES-β cells in the recipient mice was monitored by quantification of both luciferase signal and measurement of C-peptide in the blood. Graft tissue was analyzed for infiltration by TCR-Tg T cells using flow cytometry. While we did not detect any significant change in luciferase signal and C-peptide levels (FIG. 3B), we observed a profound enrichment of 1E6 T cells in ES-β grafts in both groups of recipient mice (FIG. 3C). As the C-peptide levels were not high enough for interpretation of β cell rejection, in another model, we transplanted human islets instead of ES-β cells. NSG RIP-DTR mice, which express diphtheria toxin receptor (DTR) under the control of a rat insulin promoter (RIP), were either non-reconstituted (naïve) or received human fetal thymus grafts and HSCs (HU/HU). Six weeks later, they received 2000 IEQ allogeneic human islets under the kidney capsule followed 13 weeks later by treatment with 50 ng diphtheria toxin to kill the mouse pancreatic β cells. Only HU/HU mice developed hyperglycemia (FIG. 3D) and islet grafts in HU/HU mice were heavily infiltrated with human leukocytes, including CD4 and CD8 T cells and human macrophages (FIG. 3E).

Proposed T1D model of autoimmunity. Due to current restrictions in using human fetal tissues, we have revised the models to use human cord blood HSCs, instead of fetal HSCs. As we cannot use human fetal thymus to support the generation of human T cells and the native NSG thymus is defective in positive and negative selection of human T cells, we will use human cord blood T cells from the same tissue that was used as the source of HSCs. In order to prevent graft-versus-host disease caused by cord blood T cells, we will use NSG-(Kb Db)null (IA)null mice [referred to hereafter as NSG MHC ko mice], which are deficient for both MHC class I molecule (H2-K and D) and MHC class II molecule (IA) and have shown to be resistant to GVHD (Brehm et al. 2018).

First, we will remove the native mouse thymus of NSG MHC ko mice with the method that we have developed (Khosravi-Maharlooei, et al. 2020). Then, we will sort HSCs and T cells from an HLA-A2+ cord blood sample and inject the HSCs to the recipient thymectomized and irradiated NSG MHC ko mice. T cells will be frozen for later use. Also, 2 days after injecting the mice with streptozotocin (STZ) to kill the native mouse islet β cells, we will transplant them with HLA-A2+ human islets or HLA-A2 only ES-derived β cells under the kidney capsule.

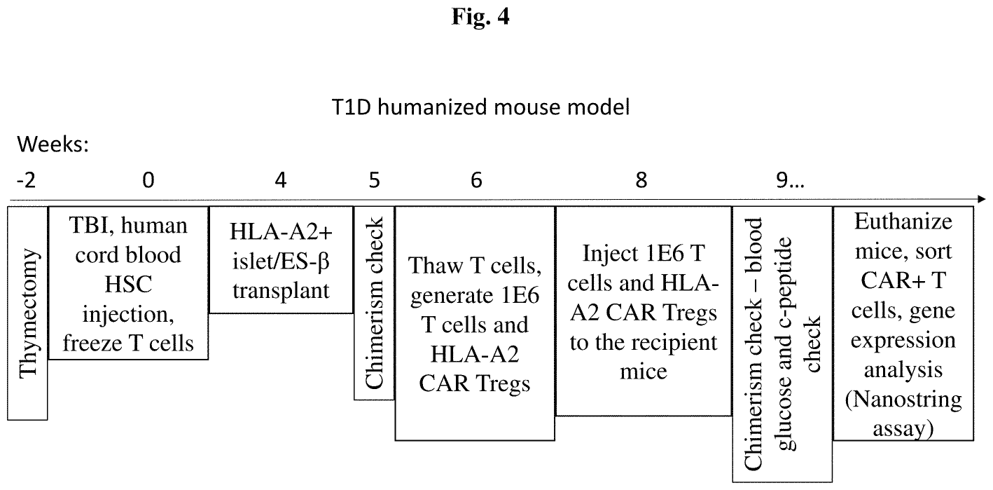

After 6 weeks, when the mice are reconstituted with cord blood-derived antigen-presenting cells, we will thaw the cord blood T cells, remove their native TCR using CRISPR, transduce them with 1E6 TCR and further expand them in vitro. We have developed a CRISPR-based strategy, which successfully removes endogenous TCR α and β chains with a very high efficiency, and is compatible with introduction of new TCR or CAR constructs. Tregs from the same cord blood sample will be transduced with each of the 4 CAR constructs shown in FIG. 1 to generate HLA-A2 CAR Tregs. 1E6 T cells and HLA-A2 CAR Tregs will be sorted and injected to the recipient mice. The recipient mice will be followed by measuring the blood glucose level every 3-4 days and a weekly check of human C-peptide level in their plasma. Also, the level of Foxp3-negative cells among reporter+ CAR Tregs will be determined using flow cytometry as an indicator of Treg conversion. At the end of the study, immune cells (including the effector and CAR T cells) are evaluated in the islet grafts and lymphoid tissues, using flow cytometry. The level of Foxp3-negative cells among reporter+ CAR Tregs are measured in different organs. Reporter+ CAR-containing T cells in different groups are sorted and their phenotype evaluated with a Nanostring CAR T cell panel, which measures mRNA levels of 780 different genes related to T cell phenotype, TCR diversity, exhaustion and metabolic and activation status. This assay will provide a cleaner picture of the phenotype of CAR Tregs after exposure to their target antigens in vivo. The best condition will be selected based on graft survival, level of CAR Treg conversion resistance and gene expression profile. FIG. 4 shows a schematic schedule of events in this proposed model.

Proposed T1D model of transplantation. In an alternative model, polyclonal non-manipulated HLA-A2-negative cord blood T cells are used as the responder T cell, instead of 1E6 TCR transduced T cells. In this system, HLA-A2+ islets/ES-β cells will be rejected by allo-reactive cord blood T cells instead of autoreactive 1E6 T cells. CAR Tregs will be generated from the same cord blood source.

B) Generation of conditioning-resistant CAR Tregs. In order to generate conditioning-resistant CAR Tregs, a guide RNA (gRNA) is designed to specifically silence human CD2 molecule using the electroporation-based system for introduction of Cas9 and gRNA (Cas9-gRNA ribonucleoprotein) (FIG. 5C). This ensures that CAR Tregs are not deleted during conditioning with anti-CD2 antibody, which we will use to deplete the recipient T cells. The best construct design selected from the above experiments will be used to generate HLA CAR Tregs. Similar in vivo studies used in A) are performed to compare the functionality of conditioning-resistant vs non-modified CAR Tregs. Humanized mice will be generated with human cord blood HSCs. A pool of autologous cord blood T cells containing one portion of T cells that undergo CRISPR-removal of CD2 and three portions of non-modified T cells will be injected at a later time point when the mice are reconstituted with human antigen presenting cells. This will ensure that after injection of anti-CD2 antibody, a T cell lymphopenic environment is created that allows for expansion of adoptively-transferred CAR Tregs, while the remaining non-modified T cells will also expand to maintain the pool of Teff cells for rejection of grafts. As explained above, HLA-A2+ human islets or HLA-A2 only ES-derived β cells will be transplanted under the kidney capsule for this model. Conditioning-resistant vs non-modified CAR Tregs are injected to the recipient mice in different groups. One dose of the anti-CD2 antibody (40) is also injected into the mice in the conditioning-resistant CAR Treg group. Graft survival and the level of infiltration of CAR Tregs in target organs will be evaluated as the readouts of this study.

C) Generation of CAR Tregs carrying immunomodulatory molecules (enhanced CAR Tregs). Once we determine the best design for the generation of conversion/conditioning-resistant CAR Tregs, we will improve the functionality of these Tregs by overexpressing the immunomodulatory molecules PD-L1 (176 amino acids (Kalscheuer et al. 2012)), TGF-β (112 amino acids (Poniatowski et al. 2015)), and CTLA-4Ig (445 amino acids (Yazdanpanah-Samani et al. 2015)) under the control of the NFAT6 minimal promoter (Chmielewski et al. 2011; Uchibori et al. 2019). Transcription of these factors will begin upon CAR-redirected T cell activation in order to target local T cells, B cells, macrophage/monocytes and DCs within the grafts. HLA-A2 CAR constructs with the lowest degree of CAR Treg conversion and the best graft survival (selected from the previous studies) are be used in these experiments. New constructs are produced with each of the 3 different immunomodulatory genes, each with a separate reporter, under the control of the NFAT6 minimal promoter (Chmielewski et al. 2011; Uchibori et al. 2019). Human Tregs are transduced with a CAR construct in addition to one of these immunomodulatory molecules.

Similar to the previous studies, in vivo models will be generated by injection of cord blood HSCs into NSG MHC ko mice, followed by adoptive transfer of 1E6 TCR-transduced autologous T cells for the T1D models. First, we will compare the functionality of non-enhanced CAR Tregs vs a pool of enhanced CAR Tregs containing all of the 3 immunomodulatory molecules. If we see an improved graft survival in the enhanced CAR Treg group, we will test CAR Tregs with each immunomodulatory molecule separately and then in different combinations to find the ideal set of immunomodulatory molecules needed for local suppression of immune system in T1D models. We have identified IL-10 (178 amino acids (Gesser et al. 1997)), IDO1 (403 amino acids (Dai and Gupta 199)) and blocking antibodies for CD40 and IFN-g as other potential immunomodulatory molecules that could be tested with this system in future.

D) Generation of “off-the-shelf” CAR Tregs. Our initial experiments will be performed with autologous Tregs to generate SLA CAR Tregs. As it will be easier, more consistent and less costly to use allogeneic Tregs, we will combine the above-mentioned engineering strategies with these strategies to generate “off-the-shelf” CAR Tregs: 1-CRISPR-based removal of endogenous TCR α and β chains, 2-CRISPR-based removal of beta-2 microglobulin (B2M) and class II, major histocompatibility complex, transactivator (CIITA), and 3-lentiviral introduction of HLA-G and HLA-E molecules. Removal of endogenous TCRs will prevent graft-versus-host disease. Removal of B2M and CIITA will prevent expression of HLA-I and II molecules on CAR Tregs and hence prevents their rejection by the recipient T cells. Lentiviral introduction of HLA-E and G will prevent rejection of CAR Tregs by the recipient NK and T cells.

### Example 2

### Combination of CAR Tregs and Bi-Specific Antibodies for Treating Autoimmune Diseases

We have developed a humanized mouse model of T1D, in which the disease is introduced by adoptively transfer of T1D-reactive TCR-transduced autologous T cells to a cohort of HLA Tg humanized mice (Tan et al. 2017). This model (T1D HU mice) allows us to evaluate the proposed designs for CAR Treg therapy in prevention and treatment of T1D.
Aim 1: Evaluate SUPRA CAR Tregs against different islet or pancreatic cells surface antigens and with different binding strengths in repression of autoimmune attack in T1D HU mice
Aim 2: Assess the combination of CAR Treg therapy with bispecific antibodies against CTLA-4 and islet surface antigens to facilitate trafficking of CAR Tregs (and other endogenous Tregs) to islets and pancreatic lymph nodes.

### Experimental Design/Methodology:

Aim 1. SUPRA CAR constructs will be generated with different targeting modules, including antibodies against islet surface antigens (Dorrell et al. 2011). We will use NSG RIP-DTR mice, which express the human diphtheria toxin receptor (DTR) driven by the rat insulin 2 promoter. As DTR is only expressed by mouse beta cells, an anti-DTR antibody would be another good candidate as a targeting module. Multiple zipper molecules with different binding affinities will be designed for each targeting module to evaluate the effect of binding strength on functionality of CAR Tregs. Humanized mice will be generated by transplantation of HLA-A2⁺ and DQ8⁺ human fetal liver CD34⁺ HSCs and autologous fetal thymus into HLA-A2 and DQ8 Tg NSG-DTR mice, as we previously described (Tan et al. 2017). This model (T1D HU mice) allows us to evaluate the proposed designs for CAR Treg therapy in prevention and treatment of T1D. One mouse in the cohort will be euthanized around 16 weeks post-transplantation. Sorted CD4 and CD8 T cells will be lentivirally transduced with T1D-reactive TCRs, expanded and adoptively transferred to the other mice in the cohort, in order to induce T1D. Likewise, sorted Treg cells (CD4⁺ CD25^{high} CD127^{low}) will be virally transduced with CAR constructs, expanded and adoptively transferred to the same mice. Different target modules against different target antigens and with different binding strength of zipper molecules will be administered and their effect in prevention of T1D will be evaluated. Additionally, the capacity of CAR Tregs in suppression of autoimmune responses will be evaluated in vitro with TCR-transduced T cells as responder cells and autologous HSC-derived DCs loaded with cognate antigens as stimulator cells.
Aim 2. Bi-specific antibodies will be generated using antibodies against islet surface antigens (described in Aim 1) and CTLA-4, which is expressed on Tregs. Selected CAR constructs from Aim 1 will be supplemented with a luciferase reporter to evaluate the effect of bi-specific antibodies in trafficking of CAR Tregs into pancreatic islets. After euthanizing the recipient mice, the proportion of endogenous Tregs within CD4 T cells will be evaluated in islets and other tissues to see whether bi-specific antibodies facilitated localization of non-CAR Tregs into the islets or not. The effect of bi-specific antibodies in prevention of T1D will be evaluated in presence and absence of CAR Tregs.

### REFERENCES

Adair et al. Human tregs made antigen specific by gene modification: The power to treat autoimmunity and antidrug antibodies with precision. Frontiers in Immunology (2017). doi:10.3389/fimmu.2017.01117
Bandgar et al. Management issues with exogenous steroid therapy. Indian J. Endocrinol. Metab. (2013). doi:10.4103/2230-8210.123548
Bluestone et al. T cells in the control of organ-specific autoimmunity. J. Clin. Invest. (2015). doi:10.1172/JCI78089
Brehm et al. Lack of acute xenogeneic graft-versus-host disease, but retention of T-cell function following engraftment of human peripheral blood mononuclear cells in NSG mice deficient in MHC class I and II expression. FASEB J. (2019). doi:10.1096/fj.201800636R
Bulek et al. Structural basis for the killing of human beta cells by CD8 + T cells in type 1 diabetes. Nat. Immunol. (2012). doi:10.1038/ni.2206
Carlotti et al. Development of an inducible suicide gene system based on human caspase 8. Cancer Gene Ther. (2005). doi:10.1038/sj.cgt.7700825
Chmielewski et al. IL-12 release by engineered T cells expressing chimeric antigen receptors can effectively muster an antigen-independent macrophage response on tumor cells that have shut down tumor antigen expression. Cancer Res. (2011). doi:10.1158/0008-5472.CAN-11-0103
Cho et al. Universal Chimeric Antigen Receptors for Multiplexed and Logical Control of T Cell Responses, Cell (2018) 173(6):1426-1438.
Dai and Gupta. Molecular cloning, sequencing and expression of human interferon-γ-inducible indoleamine 2,3-dioxygenase cDNA. Biochem. Biophys. Res. Commun. (1990). doi:10.1016/0006-291X(90)91666-G
Dawson et al. Systematic testing and specificity mapping of alloantigen-specific chimeric antigen receptors in regulatory T cells. JCI Insight (2019). doi:10.1172/jci.insight.123672
DeKoter et al. Regulation of the interleukin-7 receptor a promoter by the Ets transcription factors PU.1 and GA-binding protein in developing B cells. J. Biol. Chem. (2007) doi:10.1074/jbc.M700377200
Dorrell et al. Isolation of mouse pancreatic alpha, beta, duct and acinar populations with cell surface markers. Mol. Cell. Endocrinol. (2011) 339(1-2): 144-150
Elinav et al. Redirection of Regulatory T Cells With Predetermined Specificity for the Treatment of Experimental Colitis in Mice. Gastroenterology (2008). doi:10.1053/j.gastro.2008.02.060
Fransson et al. CAR/FoxP3-engineered T regulatory cells target the CNS and suppress EAE upon intranasal delivery. J. Neuroinflammation (2012). doi:10.1186/1742-2094-9-112
Gesser et al. Identification of functional domains on human interleukin 10. Proc. Natl. Acad. Sci. U.S.A. (1997). doi:10.1073/pnas.94.26.14620
Haddadi et al. Autoimmunity as a target for chimeric immune receptor therapy: A new vision to therapeutic potential. Blood Reviews (2020). doi:10.1016/j.blre.2019.100645
Hua. et al. Pathological conversion of regulatory T cells is associated with loss of allotolerance. Sci. Rep. (2018). doi:10.1038/s41598-018-25384-x
Kalscheuer et al. A model for personalized in vivo analysis of human immune responsiveness. Sci. Transl. Med. (2012). doi:10.1126/scitranslmed.3003481
Kawai et al. HLA-mismatched renal transplantation without maintenance immunosuppression. N. Engl. J. Med. (2008). doi:10.1056/NEJMoa071074
Khosravi-Maharlooei et al. Rapid thymectomy of NSG mice to analyze the role of native and grafted thymi in humanized mice. Eur. J. Immunol. (2020). doi:10.1002/eji.201948205
Koenen et al. Human CD25highFoxp3pos regulatory T cells differentiate into IL-17 producing cells. Blood (2008). doi:10.1182/blood-2008-01-133967
Li et al. Humanized mice reveal new insights into the thymic selection of human autoreactive CD8+ T cells. Front. Immunol. (2019). doi:10.3389/fimmu.2019.00063
Liu et al. CD127 expression inversely correlates with FoxP3 and suppressive function of human CD4+ T reg cells. J. Exp. Med. (2006). doi:10.1084/jem.20060772
Mantel et al. Molecular Mechanisms Underlying FOXP3 Induction in Human T Cells. J. Immunol. (2006). doi:10.4049/jimmunol.176.6.3593
Martin et al. Membrane oligomerization and cleavage activates the caspase-8 (FLICE/MACHα1) death signal. J. Biol. Chem. (1998). doi:10.1074/jbc.273.8.4345
MacDonald et al. Alloantigen-specific regulatory T cells generated with a chimeric antigen receptor. J. Clin. Invest. (2016). doi:10.1172/JCI82771
Mancusi et al. CD4+FOXP3+Regulatory T Cell Therapies in HLA Haploidentical Hematopoietic Transplantation. Frontiers in Immunology (2019). doi:10.3389/fimmu.2019.02901
Neelapu. CAR-T efficacy: Is conditioning the key? Blood (2019). doi:10.1182/blood-2019-03-900928
Poniatowski et al. Transforming growth factor beta family: Insight into the role of growth factors in regulation of fracture healing biology and potential clinical applications. Mediators of Inflammation (2015). doi:10.1155/2015/137823
Romano et al. Past, present, and future of regulatory T cell therapy in transplantation and autoimmunity. Frontiers in Immunology (2019). doi:10.3389/fimmu.2019.00043
Sachs. Transplantation tolerance through mixed chimerism: From allo to xeno. Xenotransplantation (2018). doi:10.1111/xen.12420
Sadlon et al. Genome-wide Identification of Human FOXP3 Target Genes Cells in Natural Regulatory T Cells. Journal of Immunology (2010) 185(2): 1071-1081.
Skuljec et al. Chimeric antigen receptor-redirected regulatory T cells suppress experimental allergic airway inflammation, a model of asthma. Front. Immunol. (2017). doi:10.3389/fimmu.2017.01125
Sharabi et al. Regulatory T cells in the treatment of disease. Nature Reviews Drug Discovery (2018). doi:10.1038/nrd.2018.148
Skowera et al. CTLs are targeted to kill β cells in patients with type 1 diabetes through recognition of a glucose-regulated preproinsulin epitope. J. Clin. Invest. (2008). doi:10.1172/JCI35449
Tan et al. Type 1 diabetes induction in humanized mice. Proc. Natl. Acad. Sci. (2017) 114 (41): 10954-10959.
Tang and Bluestone. Regulatory T-cell therapy in transplantation: Moving to the clinic. Cold Spring Harb. Perspect. Med. (2013). doi:10.1101/cshperspect.a015552
Tian et al. Gene modification strategies for next-generation CAR T cells against solid cancers. Journal of Hematology and Oncology (2020). doi:10.1186/s13045-020-00890-6
Uchibori et al. Functional Analysis of an Inducible Promoter Driven by Activation Signals from a Chimeric Antigen Receptor. Mol. Ther.—Oncolytics (2019). doi:10.1016/j.omto.2018.11.003
Yamada et al. Tolerance in xenotransplantation. Current Opinion in Organ Transplantation (2017). doi:10.1097/MOT.0000000000000466
Yazdanpanah-Samani et al. Construction of CTLA-4-ig fusion gene in pBudCE4.1 expression vector. Avicenna J. Med. Biotechnol. (2015).

1. A regulatory T (Treg) cell comprising a first nucleic acid construct encoding a chimeric antigen receptor (CAR), wherein the CAR comprises an antigen-binding region, wherein the first nucleic acid construct is operably linked to a Treg-specific promoter.

2. The Treg cell of claim 1, wherein the CAR binds to human leukocyte antigen A2 (HLA-A2).

3. The Treg cell of claim 1, wherein the Treg-specific promoter is forkhead box P3 (Foxp3) promoter.

4. The Treg cell of claim 1, wherein the Treg cell further comprises a suicide gene.

5. The Treg cell of claim 4, wherein the suicide gene is an inducible suicide gene.

6. The Treg cell of claim 4, wherein the suicide gene encodes Caspase 3, Caspase 7, Caspase 8 or Caspase 9 or a chimeric protein comprising CD8 and Caspase 8.

7. The Treg cell of claim 4, wherein the suicide gene is operably linked to an effector T cell (Teff)-specific promoter selected from the group consisting of granzyme B, perforin, IL-17, and IL-7 receptor (CD127).

8. The Treg cell of claim 1, wherein the antigen-binding region is a single-chain variable fragment (scFv) comprising a light chain variable region (V_{L}) and a heavy chain variable region (V_{H}).

9. The Treg cell of claim 1, wherein the CAR comprises a cytoplasmic signaling domain of CD3ζ.

10. The Treg cell of claim 1, wherein the Treg cell is substantially devoid of CD2.

11. The Treg cell of claim 1, wherein the Treg cell further comprises a second nucleic acid construct encoding an immunomodulatory molecule, wherein the second nucleic acid is operably linked to a nuclear factor of activated T cells (NFAT)-responsive promoter.

12. The Treg cell of claim 11, wherein the immunomodulatory molecule is selected from the group consisting of PD-L1, TGF-β, CTLA-4Ig, IL-10, IDO1, an anti-CD40 antibody, an anti-IFNγ antibody and combinations thereof.

13. The Treg cell of claim 1, wherein Treg cell further comprises a second nucleic acid construct encoding a gene that enhance the engraftment of hematopoietic stem cells (HSCs).

14. The Treg cell of claim 13, wherein the gene that enhance the engraftment of hematopoietic stem cells (HSCs) is selected from the group consisting of SCF, FLT3L, thrombopoietin, CXCL12, and combinations thereof

15. A method of inducing immune tolerance, or treating or preventing rejection, for transplantation in a subject to a graft obtained from a donor mammal, the method comprising administering the cell of claim 1 to the subject before, during or after transplantation.

16. The method of claim 15, the method further comprising administering a bispecific antibody.

17. The method of claim 16, wherein the bispecific antibody is specific to the Treg cell and a target site.

18. The method of claim 17, wherein the bispecific antibody is specific to CTLA-4 and an islet surface antigen.

19. A method of treating or preventing an autoimmune disease in a subject, the method comprising administering the cell of claim 1 to the subject.

20. The method of claim 19, wherein the autoimmune disease is Type 1 diabetes (T1D).
